SEQUENCE LISTING (222 sequences)

SEQ ID NO: 1 - protein, 19 aa, Artificial Sequence
  synthetic peptide
KCCVQYIKANSKFIGITEL

SEQ ID NO: 2 - protein, 21 aa, Artificial Sequence
  synthetic peptide
KCPYCVQYIKANSKFIGITEL

SEQ ID NO: 3 - protein, 20 aa, Artificial Sequence
  synthetic peptide
CPYCVQYIKANSKFIGITEL

SEQ ID NO: 4 - protein, 20 aa, Artificial Sequence
  synthetic peptide
KCHCVQYIKANSKFIGITEL

SEQ ID NO: 5 - protein, 20 aa, Artificial Sequence
  synthetic peptide
KCKCVQYIKANSKFIGITEL

SEQ ID NO: 6 - protein, 20 aa, Artificial Sequence
  synthetic peptide
KCRCVQYIKANSKFIGITEL

SEQ ID NO: 7 - protein, 20 aa, Artificial Sequence
  synthetic peptide
KCGCVQYIKANSKFIGITEL

SEQ ID NO: 8 - protein, 21 aa, Artificial Sequence
  synthetic peptide
KCPYCVQYIKANSKFIGITEL

SEQ ID NO: 9 - protein, 22 aa, Artificial Sequence
  synthetic peptide
KCKPYCVQYIKANSKFIGITEL

SEQ ID NO: 10 - protein, 22 aa, Artificial Sequence
  synthetic peptide
KCPKYCVQYIKANSKFIGITEL

SEQ ID NO: 11 - protein, 22 aa, Artificial Sequence
  synthetic peptide
KCPYKCVQYIKANSKFIGITEL

SEQ ID NO: 12 - protein, 21 aa, Artificial Sequence
  synthetic peptide
KCPYCVQYIKANSKFIGITEL

SEQ ID NO: 13 - protein, 22 aa, Artificial Sequence
  synthetic peptide
KCRPYCVQYIKANSKFIGITEL

SEQ ID NO: 14 - protein, 22 aa, Artificial Sequence
  synthetic peptide
KCPRYCVQYIKANSKFIGITEL

SEQ ID NO: 15 - protein, 22 aa, Artificial Sequence
  synthetic peptide
KCPYRCVQYIKANSKFIGITEL

SEQ ID NO: 16 - protein, 21 aa, Artificial Sequence
  synthetic peptide
KCPYCVQYIKANSKFIGITEL

SEQ ID NO: 17 - protein, 22 aa, Artificial Sequence
  synthetic peptide
KCHPYCVQYIKANSKFIGITEL

SEQ ID NO: 18 - protein, 22 aa, Artificial Sequence
  synthetic peptide
KCPHYCVQYIKANSKFIGITEL

SEQ ID NO: 19 - protein, 22 aa, Artificial Sequence
  synthetic peptide
KCPYHCVQYIKANSKFIGITEL

SEQ ID NO: 20 - protein, 21 aa, Artificial Sequence
  synthetic peptide
KCPYCVQYIKANSKFIGITEL

SEQ ID NO: 21 - protein, 22 aa, Artificial Sequence
  synthetic peptide
KCPYACVQYIKANSKFIGITEL

SEQ ID NO: 22 - protein, 21 aa, Artificial Sequence
  synthetic peptide
KCPYCVQYIKANSKFIGITEL

SEQ ID NO: 23 - protein, 7 aa, Artificial Sequence
  synthetic peptide
  PEPTIDE (1)..(1): F,W,Y,H or T
  misc_feature (2)..(3): Xaa can be any naturally occurring amino acid
  PEPTIDE (4)..(4): V,I,L or M
  misc_feature (5)..(6): Xaa can be any naturally occurring amino acid
  PEPTIDE (7)..(7): F,W,Y,H or T
XXXXXXX

SEQ ID NO: 24 - protein, 7 aa, Artificial Sequence
  synthetic peptide
  PEPTIDE (1)..(1): F,W,Y or H
  misc_feature (2)..(3): Xaa can be any naturally occurring amino acid
  PEPTIDE (4)..(4): V,I,L or M
  misc_feature (5)..(6): Xaa can be any naturally occurring amino acid
  PEPTIDE (7)..(7): F,W,Y or H
XXXXXXX

SEQ ID NO: 25 - protein, 7 aa, Artificial Sequence
  synthetic peptide
  PEPTIDE (1)..(1): F,W,Y or T
  misc_feature (2)..(3): Xaa can be any naturally occurring amino acid
  PEPTIDE (4)..(4): V,I,L or M
  misc_feature (5)..(6): Xaa can be any naturally occurring amino acid
  PEPTIDE (7)..(7): F,W,Y or T
XXXXXXX

SEQ ID NO: 26 - protein, 7 aa, Artificial Sequence
  synthetic peptide
  PEPTIDE (1)..(1): F,W or Y
  misc_feature (2)..(3): Xaa can be any naturally occurring amino acid
  PEPTIDE (4)..(4): V,I,L or M
  misc_feature (5)..(6): Xaa can be any naturally occurring amino acid
  PEPTIDE (7)..(7): F,W or Y
XXXXXXX

SEQ ID NO: 27 - protein, 7 aa, Artificial Sequence
  synthetic peptide
  PEPTIDE (1)..(1): F,W,Y or H
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F,W,Y or H
XXXXXXX

SEQ ID NO: 28 - protein, 7 aa, Artificial Sequence
  synthetic peptide
  PEPTIDE (1)..(1): F,W,Y,H or T
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F,W,Y, H or T
XXXXXXX

SEQ ID NO: 29 - protein, 7 aa, Artificial Sequence
  synthetic peptide
  PEPTIDE (1)..(1): F,W or Y
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F,W or Y
XXXXXXX

SEQ ID NO: 30 - protein, 4 aa, Artificial Sequence
  synthetic peptide
  PEPTIDE (1)..(1): C, S or T
  misc_feature (2)..(3): Xaa can be any naturally occurring amino acid
XXXC

SEQ ID NO: 31 - protein, 4 aa, Artificial Sequence
  synthetic peptide
  misc_feature (2)..(3): Xaa can be any naturally occurring amino acid
  PEPTIDE (4)..(4): C, S or T
CXXX

SEQ ID NO: 32 - protein, 5 aa, Artificial Sequence
  synthetic peptide
  PEPTIDE (1)..(1): C, S or T
  misc_feature (2)..(4): Xaa can be any naturally occurring amino acid
XXXXC

SEQ ID NO: 33 - protein, 6 aa, Artificial Sequence
  synthetic peptide
  PEPTIDE (1)..(1): C, S or T
  misc_feature (2)..(5): Xaa can be any naturally occurring amino acid
XXXXXC

SEQ ID NO: 34 - protein, 7 aa, Artificial Sequence
  synthetic peptide
  1PEPTIDE (1)..(1): C, S or T
  misc_feature (2)..(6): Xaa can be any naturally occurring amino acid
XXXXXXC

SEQ ID NO: 35 - protein, 8 aa, Artificial Sequence
  synthetic peptide
  PEPTIDE (1)..(1): C, S or T
  misc_feature (2)..(7): Xaa can be any naturally occurring amino acid
XXXXXXXC

SEQ ID NO: 36 - protein, 5 aa, Artificial Sequence
  synthetic peptide
  misc_feature (2)..(4): Xaa can be any naturally occurring amino acid
  PEPTIDE (5)..(5): C, S or T
CXXXX

SEQ ID NO: 37 - protein, 6 aa, Artificial Sequence
  synthetic peptide
  misc_feature (2)..(5): Xaa can be any naturally occurring amino acid
  PEPTIDE (6)..(6): C, S or T
CXXXXX

SEQ ID NO: 38 - protein, 7 aa, Artificial Sequence
  synthetic peptide
  misc_feature (2)..(6): Xaa can be any naturally occurring amino acid
  PEPTIDE (7)..(7): C, S or T
CXXXXXX

SEQ ID NO: 39 - protein, 8 aa, Artificial Sequence
  synthetic peptide
  misc_feature (2)..(7): Xaa can be any naturally occurring amino acid
  PEPTIDE (8)..(8): C, S or T
CXXXXXXX

SEQ ID NO: 40 - protein, 7 aa, Artificial Sequence
  synthetic peptide
  PEPTIDE (1)..(1): F or W
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F,W,T or H
XXXXXXX

SEQ ID NO: 41 - protein, 7 aa, Artificial Sequence
  synthetic peptide
  PEPTIDE (1)..(1): F,W,T or H
  misc_feature (2)..(3): Xaa can be any naturally occurring amino acid
  PEPTIDE (4)..(4): I,L or M
  misc_feature (5)..(6): Xaa can be any naturally occurring amino acid
  PEPTIDE (7)..(7): F or W
XXXXXXX

SEQ ID NO: 42 - protein, 5 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
  PEPTIDE (3)..(3): G, A, V, L, I, M, F, W, P, S, T, C, Y, N, Q, D, E, K, R, H, or non-natural basic amino acid
  PEPTIDE (4)..(4): G, A, V, L, I, M, F, W, P, S, T, C, Y, N, Q, D, E, K, R, H, or non-natural basic amino acid
CXXXC

SEQ ID NO: 43 - protein, 5 aa, Artificial Sequence
  synthetic peptide
  misc_feature (2)..(2): Xaa can be any naturally occurring amino acid
CXPYC

SEQ ID NO: 44 - protein, 5 aa, Artificial Sequence
  synthetic peptide
  misc_feature (3)..(3): Xaa can be any naturally occurring amino acid
CPXYC

SEQ ID NO: 45 - protein, 5 aa, Artificial Sequence
  synthetic peptide
  misc_feature (4)..(4): Xaa can be any naturally occurring amino acid
CPYXC

SEQ ID NO: 46 - protein, 5 aa, Artificial Sequence
  synthetic peptide
  misc_feature (2)..(2): Xaa can be any naturally occurring amino acid
CXHGC

SEQ ID NO: 47 - protein, 5 aa, Artificial Sequence
  synthetic peptide
  misc_feature (3)..(3): Xaa can be any naturally occurring amino acid
CHXGC

SEQ ID NO: 48 - protein, 5 aa, Artificial Sequence
  synthetic peptide
  misc_feature (4)..(4): Xaa can be any naturally occurring amino acid
CHGXC

SEQ ID NO: 49 - protein, 5 aa, Artificial Sequence
  synthetic peptide
  misc_feature (2)..(2): Xaa can be any naturally occurring amino acid
CXGPC

SEQ ID NO: 50 - protein, 5 aa, Artificial Sequence
  synthetic peptide
  misc_feature (3)..(3): Xaa can be any naturally occurring amino acid
CGXPC

SEQ ID NO: 51 - protein, 5 aa, Artificial Sequence
  synthetic peptide
  misc_feature (4)..(4): Xaa can be any naturally occurring amino acid
CGPXC

SEQ ID NO: 52 - protein, 5 aa, Artificial Sequence
  synthetic peptide
  misc_feature (2)..(2): Xaa can be any naturally occurring amino acid
CXGHC

SEQ ID NO: 53 - protein, 5 aa, Artificial Sequence
  synthetic peptide
  misc_feature (3)..(3): Xaa can be any naturally occurring amino acid
CGXHC

SEQ ID NO: 54 - protein, 5 aa, Artificial Sequence
  synthetic peptide
  misc_feature (4)..(4): Xaa can be any naturally occurring amino acid
CGHXC

SEQ ID NO: 55 - protein, 5 aa, Artificial Sequence
  synthetic peptide
  misc_feature (2)..(2): Xaa can be any naturally occurring amino acid
CXGFC

SEQ ID NO: 56 - protein, 5 aa, Artificial Sequence
  synthetic peptide
  misc_feature (3)..(3): Xaa can be any naturally occurring amino acid
CGXFC

SEQ ID NO: 57 - protein, 5 aa, Artificial Sequence
  synthetic peptide
  misc_feature (4)..(4): Xaa can be any naturally occurring amino acid
CGFXC

SEQ ID NO: 58 - protein, 5 aa, Artificial Sequence
  synthetic peptide
  misc_feature (2)..(2): Xaa can be any naturally occurring amino acid
CXRLC

SEQ ID NO: 59 - protein, 5 aa, Artificial Sequence
  synthetic peptide
  misc_feature (3)..(3): Xaa can be any naturally occurring amino acid
CRXLC

SEQ ID NO: 60 - protein, 5 aa, Artificial Sequence
  CRLXC
  misc_feature (4)..(4): Xaa can be any naturally occurring amino acid
CRLXC

SEQ ID NO: 61 - protein, 5 aa, Artificial Sequence
  synthetic peptide
  misc_feature (2)..(2): Xaa can be any naturally occurring amino acid
CXHPC

SEQ ID NO: 62 - protein, 5 aa, Artificial Sequence
  synthetic peptide
  misc_feature (3)..(3): Xaa can be any naturally occurring amino acid
CHXPC

SEQ ID NO: 63 - protein, 5 aa, Artificial Sequence
  synthetic peptide
  misc_feature (4)..(4): Xaa can be any naturally occurring amino acid
CHPXC

SEQ ID NO: 64 - protein, 6 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
  PEPTIDE (3)..(3): G, A, V, L, I, M, F, W, P, S, T, C, Y, N, Q, D, E, K, R, H, or non-natural basic amino acid
  PEPTIDE (4)..(4): G, A, V, L, I, M, F, W, P, S, T, C, Y, N, Q, D, E, K, R, H, or non-natural basic amino acid
  PEPTIDE (5)..(5): G, A, V, L, I, M, F, W, P, S, T, C, Y, N, Q, D, E, K, R, H, or non-natural basic amino acid
CXXXXC

SEQ ID NO: 65 - protein, 6 aa, Artificial Sequence
  synthetic peptide
CLAVLC

SEQ ID NO: 66 - protein, 6 aa, Artificial Sequence
  synthetic peptide
CTVQAC

SEQ ID NO: 67 - protein, 6 aa, Artificial Sequence
  synthetic peptide
CGAVHC

SEQ ID NO: 68 - protein, 6 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
CXAVLC

SEQ ID NO: 69 - protein, 6 aa, Artificial Sequence
  synthetic peptide
  PEPTIDE (3)..(3): G, A, V, L, I, M, F, W, P, S, T, C, Y, N, Q, D, E, K, R, H, or non-natural basic amino acid
CLXVLC

SEQ ID NO: 70 - protein, 6 aa, Artificial Sequence
  synthetic peptide
  PEPTIDE (4)..(4): G, A, V, L, I, M, F, W, P, S, T, C, Y, N, Q, D, E, K, R, H, or non-natural basic amino acid
CLAXLC

SEQ ID NO: 71 - protein, 6 aa, Artificial Sequence
  synthetic peptide
  PEPTIDE (5)..(5): G, A, V, L, I, M, F, W, P, S, T, C, Y, N, Q, D, E, K, R, H, or non-natural basic amino acid
CLAVXC

SEQ ID NO: 72 - protein, 6 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
CXVQAC

SEQ ID NO: 73 - protein, 6 aa, Artificial Sequence
  synthetic peptide
  PEPTIDE (3)..(3): G, A, V, L, I, M, F, W, P, S, T, C, Y, N, Q, D, E, K, R, H, or non-natural basic amino acid
CTXQAC

SEQ ID NO: 74 - protein, 6 aa, Artificial Sequence
  synthetic peptide
  PEPTIDE (4)..(4): G, A, V, L, I, M, F, W, P, S, T, C, Y, N, Q, D, E, K, R, H, or non-natural basic amino acid
CTVXAC

SEQ ID NO: 75 - protein, 6 aa, Artificial Sequence
  synthetic peptide
  PEPTIDE (5)..(5): G, A, V, L, I, M, F, W, P, S, T, C, Y, N, Q, D, E, K, R, H, or non-natural basic amino acid
CTVQXC

SEQ ID NO: 76 - protein, 6 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
CXAVHC

SEQ ID NO: 77 - protein, 6 aa, Artificial Sequence
  synthetic peptide
  PEPTIDE (3)..(3): G, A, V, L, I, M, F, W, P, S, T, C, Y, N, Q, D, E, K, R, H, or non-natural basic amino acid
CGXVHC

SEQ ID NO: 78 - protein, 6 aa, Artificial Sequence
  synthetic peptide
  PEPTIDE (4)..(4): G, A, V, L, I, M, F, W, P, S, T, C, Y, N, Q, D, E, K, R, H, or non-natural basic amino acid
CGAXHC

SEQ ID NO: 79 - protein, 6 aa, Artificial Sequence
  synthetic peptide
  PEPTIDE (5)..(5): G, A, V, L, I, M, F, W, P, S, T, C, Y, N, Q, D, E, K, R, H, or non-natural basic amino acid
CGAVXC

SEQ ID NO: 80 - protein, 7 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
  PEPTIDE (3)..(3): G, A, V, L, I, M, F, W, P, S, T, C, Y, N, Q, D, E, K, R, H, or non-natural basic amino acid
  PEPTIDE (4)..(4): G, A, V, L, I, M, F, W, P, S, T, C, Y, N, Q, D, E, K, R, H, or non-natural basic amino acid
  PEPTIDE (5)..(5): G, A, V, L, I, M, F, W, P, S, T, C, Y, N, Q, D, E, K, R, H, or non-natural basic amino acid
  PEPTIDE (6)..(6): G, A, V, L, I, M, F, W, P, S, T, C, Y, N, Q, D, E, K, R, H, or non-natural basic amino acid
CXXXXXC

SEQ ID NO: 81 - protein, 7 aa, Artificial Sequence
  synthetic peptide
CPAFPLC

SEQ ID NO: 82 - protein, 7 aa, Artificial Sequence
  synthetic peptide
CDQGGEC

SEQ ID NO: 83 - protein, 7 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
CXAFPLC

SEQ ID NO: 84 - protein, 7 aa, Artificial Sequence
  synthetic peptide
  PEPTIDE (3)..(3): G, A, V, L, I, M, F, W, P, S, T, C, Y, N, Q, D, E, K, R, H, or non-natural basic amino acid
CPXFPLC

SEQ ID NO: 85 - protein, 7 aa, Artificial Sequence
  synthetic peptide
  PEPTIDE (4)..(4): G, A, V, L, I, M, F, W, P, S, T, C, Y, N, Q, D, E, K, R, H, or non-natural basic amino acid
CPAXPLC

SEQ ID NO: 86 - protein, 7 aa, Artificial Sequence
  synthetic peptide
  PEPTIDE (5)..(5): G, A, V, L, I, M, F, W, P, S, T, C, Y, N, Q, D, E, K, R, H, or non-natural basic amino acid
CPAFXLC

SEQ ID NO: 87 - protein, 7 aa, Artificial Sequence
  synthetic peptide
  PEPTIDE (6)..(6): G, A, V, L, I, M, F, W, P, S, T, C, Y, N, Q, D, E, K, R, H, or non-natural basic amino acid
CPAFPXC

SEQ ID NO: 88 - protein, 7 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
CXQGGEC

SEQ ID NO: 89 - protein, 7 aa, Artificial Sequence
  synthetic peptide
  PEPTIDE (3)..(3): G, A, V, L, I, M, F, W, P, S, T, C, Y, N, Q, D, E, K, R, H, or non-natural basic amino acid
CDXGGEC

SEQ ID NO: 90 - protein, 7 aa, Artificial Sequence
  synthetic peptide
  PEPTIDE (4)..(4): G, A, V, L, I, M, F, W, P, S, T, C, Y, N, Q, D, E, K, R, H, or non-natural basic amino acid
CDQXGEC

SEQ ID NO: 91 - protein, 7 aa, Artificial Sequence
  synthetic peptide
  PEPTIDE (5)..(5): G, A, V, L, I, M, F, W, P, S, T, C, Y, N, Q, D, E, K, R, H, or non-natural basic amino acid
CDQGXEC

SEQ ID NO: 92 - protein, 7 aa, Artificial Sequence
  synthetic peptide
  PEPTIDE (6)..(6): G, A, V, L, I, M, F, W, P, S, T, C, Y, N, Q, D, E, K, R, H, or non-natural basic amino acid
CDQGGXC

SEQ ID NO: 93 - protein, 8 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
  PEPTIDE (3)..(3): G, A, V, L, I, M, F, W, P, S, T, C, Y, N, Q, D, E, K, R, H, or non-natural basic amino acid
  PEPTIDE (4)..(4): G, A, V, L, I, M, F, W, P, S, T, C, Y, N, Q, D, E, K, R, H, or non-natural basic amino acid
  PEPTIDE (5)..(5): G, A, V, L, I, M, F, W, P, S, T, C, Y, N, Q, D, E, K, R, H, or non-natural basic amino acid
  PEPTIDE (6)..(6): G, A, V, L, I, M, F, W, P, S, T, C, Y, N, Q, D, E, K, R, H, or non-natural basic amino acid
  PEPTIDE (7)..(7): G, A, V, L, I, M, F, W, P, S, T, C, Y, N, Q, D, E, K, R, H, or non-natural basic amino acid
CXXXXXXC

SEQ ID NO: 94 - protein, 8 aa, Artificial Sequence
  synthetic peptide
CDIADKYC

SEQ ID NO: 95 - protein, 8 aa, Artificial Sequence
  synthetic peptide
  PEPTIDE (2)..(2): G, A, V, L, I, M, F, W, P, S, T, C, Y, N, Q, D, E, K, R, H, or non-natural basic amino acid
CXIADKYC

SEQ ID NO: 96 - protein, 8 aa, Artificial Sequence
  synthetic peptide
  PEPTIDE (3)..(3): G, A, V, L, I, M, F, W, P, S, T, C, Y, N, Q, D, E, K, R, H, or non-natural basic amino acid
CDXADKYC

SEQ ID NO: 97 - protein, 8 aa, Artificial Sequence
  synthetic peptide
  PEPTIDE (4)..(4): G, A, V, L, I, M, F, W, P, S, T, C, Y, N, Q, D, E, K, R, H, or non-natural basic amino acid
CDIXDKYC

SEQ ID NO: 98 - protein, 8 aa, Artificial Sequence
  synthetic peptide
  PEPTIDE (5)..(5): G, A, V, L, I, M, F, W, P, S, T, C, Y, N, Q, D, E, K, R, H, or non-natural basic amino acid
CDIAXKYC

SEQ ID NO: 99 - protein, 8 aa, Artificial Sequence
  synthetic peptide
  PEPTIDE (6)..(6): G, A, V, L, I, M, F, W, P, S, T, C, Y, N, Q, D, E, K, R, H, or non-natural basic amino acid
CDIADXYC

SEQ ID NO: 100 - protein, 8 aa, Artificial Sequence
  synthetic peptide
  PEPTIDE (7)..(7): G, A, V, L, I, M, F, W, P, S, T, C, Y, N, Q, D, E, K, R, H, or non-natural basic amino acid
CDIADKXC

SEQ ID NO: 101 - protein, 4 aa, Artificial Sequence
  synthetic peptide
  PEPTIDE (1)..(1): basic amino acid
  PEPTIDE (2)..(2): C, S or T
  misc_feature (3)..(3): Xaa can be any naturally occurring amino acid
XXXC

SEQ ID NO: 102 - protein, 6 aa, Artificial Sequence
  synthetic peptide
  PEPTIDE (1)..(1): basic amino acid
  PEPTIDE (2)..(2): C, S or T
  misc_feature (3)..(5): Xaa can be any naturally occurring amino acid
XXXXXC

SEQ ID NO: 103 - protein, 7 aa, Artificial Sequence
  synthetic peptide
  PEPTIDE (1)..(1): basic amino acid
  PEPTIDE (2)..(2): C, S or T
  misc_feature (3)..(6): Xaa can be any naturally occurring amino acid
XXXXXXC

SEQ ID NO: 104 - protein, 8 aa, Artificial Sequence
  synthetic peptide
  PEPTIDE (1)..(1): basic amino acid
  PEPTIDE (2)..(2): C, S or T
  misc_feature (3)..(7): Xaa can be any naturally occurring amino acid
XXXXXXXC

SEQ ID NO: 105 - protein, 9 aa, Artificial Sequence
  synthetic peptide
  PEPTIDE (1)..(1): basic amino acid
  PEPTIDE (2)..(2): C, S or T
  misc_feature (3)..(8): Xaa can be any naturally occurring amino acid
XXXXXXXXC

SEQ ID NO: 106 - protein, 4 aa, Artificial Sequence
  synthetic peptide
  PEPTIDE (1)..(1): basic amino acid
  misc_feature (2)..(2): Xaa can be any naturally occurring amino acid
  PEPTIDE (3)..(3): C, S or T
XXXC

SEQ ID NO: 107 - protein, 5 aa, Artificial Sequence
  synthetic peptide
  PEPTIDE (1)..(1): basic amino acid
  misc_feature (2)..(2): Xaa can be any naturally occurring amino acid
  PEPTIDE (3)..(3): C, S or T
  misc_feature (4)..(4): Xaa can be any naturally occurring amino acid
XXXXC

SEQ ID NO: 108 - protein, 7 aa, Artificial Sequence
  synthetic peptide
  PEPTIDE (1)..(1): basic amino acid
  misc_feature (2)..(2): Xaa can be any naturally occurring amino acid
  PEPTIDE (3)..(3): C, S or T
  misc_feature (4)..(6): Xaa can be any naturally occurring amino acid
XXXXXXC

SEQ ID NO: 109 - protein, 8 aa, Artificial Sequence
  synthetic peptide
  PEPTIDE (1)..(1): basic amino acid
  misc_feature (2)..(2): Xaa can be any naturally occurring amino acid
  PEPTIDE (3)..(3): C, S or T
  misc_feature (4)..(7): Xaa can be any naturally occurring amino acid
XXXXXXXC

SEQ ID NO: 110 - protein, 9 aa, Artificial Sequence
  synthetic peptide
  PEPTIDE (1)..(1): basic amino acid
  misc_feature (2)..(2): Xaa can be any naturally occurring amino acid
  PEPTIDE (3)..(3): C, S or T
  misc_feature (4)..(8): Xaa can be any naturally occurring amino acid
XXXXXXXXC

SEQ ID NO: 111 - protein, 10 aa, Artificial Sequence
  synthetic peptide
  PEPTIDE (1)..(1): basic amino acid
  misc_feature (2)..(2): Xaa can be any naturally occurring amino acid
  PEPTIDE (3)..(3): C, S or T
  misc_feature (4)..(9): Xaa can be any naturally occurring amino acid
XXXXXXXXXC

SEQ ID NO: 112 - protein, 5 aa, Artificial Sequence
  synthetic peptide
  PEPTIDE (1)..(1): basic amino acid
  misc_feature (2)..(3): Xaa can be any naturally occurring amino acid
  PEPTIDE (4)..(4): C, S or T
XXXXC

SEQ ID NO: 113 - protein, 6 aa, Artificial Sequence
  synthetic peptide
  PEPTIDE (1)..(1): basic amino acid
  misc_feature (2)..(3): Xaa can be any naturally occurring amino acid
  PEPTIDE (4)..(4): C, S or T
  misc_feature (5)..(5): Xaa can be any naturally occurring amino acid
XXXXXC

SEQ ID NO: 114 - protein, 8 aa, Artificial Sequence
  synthetic peptide
  PEPTIDE (1)..(1): basic amino acid
  misc_feature (2)..(3): Xaa can be any naturally occurring amino acid
  PEPTIDE (4)..(4): C, S or T
  misc_feature (5)..(7): Xaa can be any naturally occurring amino acid
XXXXXXXC

SEQ ID NO: 115 - protein, 8 aa, Artificial Sequence
  synthetic peptide
  PEPTIDE (1)..(1): basic amino acid
  misc_feature (2)..(3): Xaa can be any naturally occurring amino acid
  PEPTIDE (4)..(4): C, S or T
  misc_feature (5)..(7): Xaa can be any naturally occurring amino acid
XXXXXXXC

SEQ ID NO: 116 - protein, 10 aa, Artificial Sequence
  synthetic peptide
  PEPTIDE (1)..(1): basic amino acid
  misc_feature (2)..(3): Xaa can be any naturally occurring amino acid
  PEPTIDE (4)..(4): C, S or T
  misc_feature (5)..(9): Xaa can be any naturally occurring amino acid
XXXXXXXXXC

SEQ ID NO: 117 - protein, 11 aa, Artificial Sequence
  synthetic peptide
  PEPTIDE (1)..(1): basic amino acid
  misc_feature (2)..(3): Xaa can be any naturally occurring amino acid
  PEPTIDE (4)..(4): C, S or T
  misc_feature (5)..(10): Xaa can be any naturally occurring amino acid
XXXXXXXXXXC

SEQ ID NO: 118 - protein, 6 aa, Artificial Sequence
  synthetic peptide
  PEPTIDE (1)..(1): basic amino acid
  misc_feature (2)..(4): Xaa can be any naturally occurring amino acid
  PEPTIDE (5)..(5): C, S or T
XXXXXC

SEQ ID NO: 119 - protein, 7 aa, Artificial Sequence
  synthetic peptide
  PEPTIDE (1)..(1): basic amino acid
  misc_feature (2)..(4): Xaa can be any naturally occurring amino acid
  PEPTIDE (5)..(5): C, S or T
  misc_feature (6)..(6): Xaa can be any naturally occurring amino acid
XXXXXXC

SEQ ID NO: 120 - protein, 9 aa, Artificial Sequence
  synthetic peptide
  PEPTIDE (1)..(1): basic amino acid
  misc_feature (2)..(4): Xaa can be any naturally occurring amino acid
  PEPTIDE (5)..(5): C, S or T
  misc_feature (6)..(8): Xaa can be any naturally occurring amino acid
XXXXXXXXC

SEQ ID NO: 121 - protein, 10 aa, Artificial Sequence
  synthetic peptide
  PEPTIDE (1)..(1): basic amino acid
  misc_feature (2)..(4): Xaa can be any naturally occurring amino acid
  PEPTIDE (5)..(5): C, S or T
  misc_feature (6)..(9): Xaa can be any naturally occurring amino acid
XXXXXXXXXC

SEQ ID NO: 122 - protein, 11 aa, Artificial Sequence
  synthetic peptide
  PEPTIDE (1)..(1): basic amino acid
  misc_feature (2)..(4): Xaa can be any naturally occurring amino acid
  PEPTIDE (5)..(5): C, S or T
  misc_feature (6)..(10): Xaa can be any naturally occurring amino acid
XXXXXXXXXXC

SEQ ID NO: 123 - protein, 12 aa, Artificial Sequence
  synthetic peptide
  PEPTIDE (1)..(1): basic amino acid
  misc_feature (2)..(4): Xaa can be any naturally occurring amino acid
  PEPTIDE (5)..(5): C, S or T
  misc_feature (6)..(11): Xaa can be any naturally occurring amino acid
XXXXXXXXXXXC

SEQ ID NO: 124 - protein, 4 aa, Artificial Sequence
  synthetic peptide
  PEPTIDE (1)..(1): C, S or T
  misc_feature (2)..(2): Xaa can be any naturally occurring amino acid
  PEPTIDE (4)..(4): basic amino acid
XXCX

SEQ ID NO: 125 - protein, 6 aa, Artificial Sequence
  synthetic peptide
  PEPTIDE (1)..(1): C, S or T
  misc_feature (2)..(4): Xaa can be any naturally occurring amino acid
  PEPTIDE (6)..(6): basic amino acid
XXXXCX

SEQ ID NO: 126 - protein, 7 aa, Artificial Sequence
  synthetic peptide
  PEPTIDE (1)..(1): C, S or T
  misc_feature (2)..(5): Xaa can be any naturally occurring amino acid
  PEPTIDE (7)..(7): basic amino acid
XXXXXCX

SEQ ID NO: 127 - protein, 8 aa, Artificial Sequence
  synthetic peptide
  PEPTIDE (1)..(1): C, S or T
  misc_feature (2)..(6): Xaa can be any naturally occurring amino acid
  PEPTIDE (8)..(8): basic amino acid
XXXXXXCX

SEQ ID NO: 128 - protein, 9 aa, Artificial Sequence
  synthetic peptide
  PEPTIDE (1)..(1): C, S or T
  misc_feature (2)..(7): Xaa can be any naturally occurring amino acid
  PEPTIDE (9)..(9): basic amino acid
XXXXXXXCX

SEQ ID NO: 129 - protein, 4 aa, Artificial Sequence
  synthetic peptide
  PEPTIDE (1)..(1): C, S or T
  misc_feature (3)..(3): Xaa can be any naturally occurring amino acid
  PEPTIDE (4)..(4): basic amino acid
XCXX

SEQ ID NO: 130 - protein, 5 aa, Artificial Sequence
  synthetic peptide
  PEPTIDE (1)..(1): C, S or T
  misc_feature (2)..(2): Xaa can be any naturally occurring amino acid
  misc_feature (4)..(4): Xaa can be any naturally occurring amino acid
  PEPTIDE (5)..(5): basic amino acid
XXCXX

SEQ ID NO: 131 - protein, 7 aa, Artificial Sequence
  synthetic peptide
  PEPTIDE (1)..(1): C, S or T
  misc_feature (2)..(4): Xaa can be any naturally occurring amino acid
  misc_feature (6)..(6): Xaa can be any naturally occurring amino acid
  PEPTIDE (7)..(7): basic amino acid
XXXXCXX

SEQ ID NO: 132 - protein, 8 aa, Artificial Sequence
  synthetic peptide
  PEPTIDE (1)..(1): C, S or T
  misc_feature (2)..(5): Xaa can be any naturally occurring amino acid
  misc_feature (7)..(7): Xaa can be any naturally occurring amino acid
  PEPTIDE (8)..(8): basic amino acid
XXXXXCXX

SEQ ID NO: 133 - protein, 9 aa, Artificial Sequence
  synthetic peptide
  PEPTIDE (1)..(1): C, S or T
  misc_feature (2)..(6): Xaa can be any naturally occurring amino acid
  misc_feature (8)..(8): Xaa can be any naturally occurring amino acid
  PEPTIDE (9)..(9): basic amino acid
XXXXXXCXX

SEQ ID NO: 134 - protein, 10 aa, Artificial Sequence
  synthetic peptide
  PEPTIDE (1)..(1): C, S or T
  misc_feature (2)..(7): Xaa can be any naturally occurring amino acid
  misc_feature (9)..(9): Xaa can be any naturally occurring amino acid
  PEPTIDE (10)..(10): basic amino acid
XXXXXXXCXX

SEQ ID NO: 135 - protein, 5 aa, Artificial Sequence
  synthetic peptide
  PEPTIDE (1)..(1): C, S or T
  misc_feature (3)..(4): Xaa can be any naturally occurring amino acid
  PEPTIDE (5)..(5): basic amino acid
XCXXX

SEQ ID NO: 136 - protein, 6 aa, Artificial Sequence
  synthetic peptide
  PEPTIDE (1)..(1): C, S or T
  misc_feature (2)..(2): Xaa can be any naturally occurring amino acid
  misc_feature (4)..(5): Xaa can be any naturally occurring amino acid
  PEPTIDE (6)..(6): basic amino acid
XXCXXX

SEQ ID NO: 137 - protein, 8 aa, Artificial Sequence
  synthetic peptide
  PEPTIDE (1)..(1): C, S or T
  misc_feature (2)..(4): Xaa can be any naturally occurring amino acid
  misc_feature (6)..(7): Xaa can be any naturally occurring amino acid
  PEPTIDE (8)..(8): basic amino acid
XXXXCXXX

SEQ ID NO: 138 - protein, 9 aa, Artificial Sequence
  synthetic peptide
  PEPTIDE (1)..(1): C, S or T
  misc_feature (2)..(5): Xaa can be any naturally occurring amino acid
  misc_feature (7)..(8): Xaa can be any naturally occurring amino acid
  PEPTIDE (9)..(9): basic amino acid
XXXXXCXXX

SEQ ID NO: 139 - protein, 10 aa, Artificial Sequence
  synthetic peptide
  PEPTIDE (1)..(1): C, S or T
  misc_feature (2)..(6): Xaa can be any naturally occurring amino acid
  misc_feature (8)..(9): Xaa can be any naturally occurring amino acid
  PEPTIDE (10)..(10): basic amino acid
XXXXXXCXXX

SEQ ID NO: 140 - protein, 11 aa, Artificial Sequence
  synthetic peptide
  PEPTIDE (1)..(1): C, S or T
  misc_feature (2)..(7): Xaa can be any naturally occurring amino acid
  misc_feature (9)..(10): Xaa can be any naturally occurring amino acid
  PEPTIDE (11)..(11): basic amino acid
XXXXXXXCXXX

SEQ ID NO: 141 - protein, 6 aa, Artificial Sequence
  synthetic peptide
  PEPTIDE (1)..(1): C, S or T
  misc_feature (3)..(5): Xaa can be any naturally occurring amino acid
  PEPTIDE (6)..(6): basic amino acid
XCXXXX

SEQ ID NO: 142 - protein, 7 aa, Artificial Sequence
  synthetic peptide
  PEPTIDE (1)..(1): C, S or T
  misc_feature (2)..(2): Xaa can be any naturally occurring amino acid
  misc_feature (4)..(6): Xaa can be any naturally occurring amino acid
  PEPTIDE (7)..(7): basic amino acid
XXCXXXX

SEQ ID NO: 143 - protein, 9 aa, Artificial Sequence
  synthetic peptide
  PEPTIDE (1)..(1): C, S or T
  misc_feature (2)..(4): Xaa can be any naturally occurring amino acid
  misc_feature (6)..(8): Xaa can be any naturally occurring amino acid
  PEPTIDE (9)..(9): basic amino acid
XXXXCXXXX

SEQ ID NO: 144 - protein, 10 aa, Artificial Sequence
  synthetic peptide
  PEPTIDE (1)..(1): C, S or T
  misc_feature (2)..(5): Xaa can be any naturally occurring amino acid
  misc_feature (7)..(9): Xaa can be any naturally occurring amino acid
  PEPTIDE (10)..(10): basic amino acid
XXXXXCXXXX

SEQ ID NO: 145 - protein, 11 aa, Artificial Sequence
  synthetic peptide
  PEPTIDE (1)..(1): C, S or T
  misc_feature (2)..(6): Xaa can be any naturally occurring amino acid
  misc_feature (8)..(10): Xaa can be any naturally occurring amino acid
  PEPTIDE (11)..(11): basic amino acid
XXXXXXCXXXX

SEQ ID NO: 146 - protein, 12 aa, Artificial Sequence
  synthetic peptide
  PEPTIDE (1)..(1): C, S or T
  misc_feature (2)..(7): Xaa can be any naturally occurring amino acid
  misc_feature (9)..(11): Xaa can be any naturally occurring amino acid
  PEPTIDE (12)..(12): basic amino acid
XXXXXXXCXXXX

SEQ ID NO: 147 - protein, 4 aa, Artificial Sequence
  synthetic peptide
  PEPTIDE (1)..(1): basic amino acid
  misc_feature (3)..(3): Xaa can be any naturally occurring amino acid
  PEPTIDE (4)..(4): C, S or T
XCXX

SEQ ID NO: 148 - protein, 6 aa, Artificial Sequence
  synthetic peptide
  PEPTIDE (1)..(1): basic amino acid
  misc_feature (3)..(5): Xaa can be any naturally occurring amino acid
  PEPTIDE (6)..(6): C, S or T
XCXXXX

SEQ ID NO: 149 - protein, 7 aa, Artificial Sequence
  synthetic peptide
  PEPTIDE (1)..(1): basic amino acid
  misc_feature (3)..(6): Xaa can be any naturally occurring amino acid
  PEPTIDE (7)..(7): C, S or T
XCXXXXX

SEQ ID NO: 150 - protein, 8 aa, Artificial Sequence
  synthetic peptide
  PEPTIDE (1)..(1): basic amino acid
  misc_feature (3)..(7): Xaa can be any naturally occurring amino acid
  PEPTIDE (8)..(8): C, S or T
XCXXXXXX

SEQ ID NO: 151 - protein, 9 aa, Artificial Sequence
  synthetic peptide
  PEPTIDE (1)..(1): basic amino acid
  misc_feature (3)..(8): Xaa can be any naturally occurring amino acid
  PEPTIDE (9)..(9): C, S or T
XCXXXXXXX

SEQ ID NO: 152 - protein, 4 aa, Artificial Sequence
  synthetic peptide
  PEPTIDE (1)..(1): basic amino acid
  misc_feature (2)..(2): Xaa can be any naturally occurring amino acid
  PEPTIDE (4)..(4): C, S or T
XXCX

SEQ ID NO: 153 - protein, 5 aa, Artificial Sequence
  synthetic peptide
  PEPTIDE (1)..(1): basic amino acid
  misc_feature (2)..(2): Xaa can be any naturally occurring amino acid
  misc_feature (4)..(4): Xaa can be any naturally occurring amino acid
  PEPTIDE (5)..(5): C, S or T
XXCXX

SEQ ID NO: 154 - protein, 7 aa, Artificial Sequence
  synthetic peptide
  PEPTIDE (1)..(1): basic amino acid
  misc_feature (2)..(2): Xaa can be any naturally occurring amino acid
  misc_feature (4)..(6): Xaa can be any naturally occurring amino acid
  PEPTIDE (7)..(7): C, S or T
XXCXXXX

SEQ ID NO: 155 - protein, 8 aa, Artificial Sequence
  synthetic peptide
  PEPTIDE (1)..(1): basic amino acid
  misc_feature (2)..(2): Xaa can be any naturally occurring amino acid
  misc_feature (4)..(7): Xaa can be any naturally occurring amino acid
  PEPTIDE (8)..(8): C, S or T
XXCXXXXX

SEQ ID NO: 156 - protein, 9 aa, Artificial Sequence
  synthetic peptide
  PEPTIDE (1)..(1): basic amino acid
  misc_feature (2)..(2): Xaa can be any naturally occurring amino acid
  misc_feature (4)..(8): Xaa can be any naturally occurring amino acid
  PEPTIDE (9)..(9): C, S or T
XXCXXXXXX

SEQ ID NO: 157 - protein, 10 aa, Artificial Sequence
  synthetic peptide
  PEPTIDE (1)..(1): basic amino acid
  misc_feature (2)..(2): Xaa can be any naturally occurring amino acid
  misc_feature (4)..(9): Xaa can be any naturally occurring amino acid
  PEPTIDE (10)..(10): C, S or T
XXCXXXXXXX

SEQ ID NO: 158 - protein, 5 aa, Artificial Sequence
  synthetic peptide
  PEPTIDE (1)..(1): basic amino acid
  misc_feature (2)..(3): Xaa can be any naturally occurring amino acid
  PEPTIDE (5)..(5): C, S or T
XXXCX

SEQ ID NO: 159 - protein, 6 aa, Artificial Sequence
  synthetic peptide
  PEPTIDE (1)..(1): basic amino acid
  misc_feature (2)..(3): Xaa can be any naturally occurring amino acid
  misc_feature (5)..(5): Xaa can be any naturally occurring amino acid
  PEPTIDE (6)..(6): C, S or T
XXXCXX

SEQ ID NO: 160 - protein, 8 aa, Artificial Sequence
  synthetic peptide
  PEPTIDE (1)..(1): basic amino acid
  misc_feature (2)..(3): Xaa can be any naturally occurring amino acid
  misc_feature (5)..(7): Xaa can be any naturally occurring amino acid
  PEPTIDE (8)..(8): C, S or T
XXXCXXXX

SEQ ID NO: 161 - protein, 9 aa, Artificial Sequence
  synthetic peptide
  PEPTIDE (1)..(1): basic amino acid
  misc_feature (2)..(3): Xaa can be any naturally occurring amino acid
  misc_feature (5)..(8): Xaa can be any naturally occurring amino acid
  PEPTIDE (9)..(9): C, S or T
XXXCXXXXX

SEQ ID NO: 162 - protein, 10 aa, Artificial Sequence
  synthetic peptide
  PEPTIDE (1)..(1): basic amino acid
  misc_feature (2)..(3): Xaa can be any naturally occurring amino acid
  misc_feature (5)..(9): Xaa can be any naturally occurring amino acid
  PEPTIDE (10)..(10): C, S or T
XXXCXXXXXX

SEQ ID NO: 163 - protein, 11 aa, Artificial Sequence
  synthetic peptide
  PEPTIDE (1)..(1): basic amino acid
  misc_feature (2)..(3): Xaa can be any naturally occurring amino acid
  misc_feature (5)..(10): Xaa can be any naturally occurring amino acid
  PEPTIDE (11)..(11): C, S or T
XXXCXXXXXXX

SEQ ID NO: 164 - protein, 6 aa, Artificial Sequence
  synthetic peptide
  PEPTIDE (1)..(1): basic amino acid
  misc_feature (2)..(4): Xaa can be any naturally occurring amino acid
  PEPTIDE (6)..(6): C, S or T
XXXXCX

SEQ ID NO: 165 - protein, 7 aa, Artificial Sequence
  synthetic peptide
  PEPTIDE (1)..(1): basic amino acid
  misc_feature (2)..(4): Xaa can be any naturally occurring amino acid
  misc_feature (6)..(6): Xaa can be any naturally occurring amino acid
  PEPTIDE (7)..(7): C, S or T
XXXXCXX

SEQ ID NO: 166 - protein, 9 aa, Artificial Sequence
  synthetic peptide
  PEPTIDE (1)..(1): basic amino acid
  misc_feature (2)..(4): Xaa can be any naturally occurring amino acid
  misc_feature (6)..(8): Xaa can be any naturally occurring amino acid
  PEPTIDE (9)..(9): C, S or T
XXXXCXXXX

SEQ ID NO: 167 - protein, 10 aa, Artificial Sequence
  synthetic peptide
  PEPTIDE (1)..(1): basic amino acid
  misc_feature (2)..(4): Xaa can be any naturally occurring amino acid
  misc_feature (6)..(9): Xaa can be any naturally occurring amino acid
  PEPTIDE (10)..(10): C, S or T
XXXXCXXXXX

SEQ ID NO: 168 - protein, 11 aa, Artificial Sequence
  synthetic peptide
  PEPTIDE (1)..(1): basic amino acid
  misc_feature (2)..(4): Xaa can be any naturally occurring amino acid
  misc_feature (6)..(10): Xaa can be any naturally occurring amino acid
  PEPTIDE (11)..(11): C, S or T
XXXXCXXXXXX

SEQ ID NO: 169 - protein, 12 aa, Artificial Sequence
  synthetic peptide
  PEPTIDE (1)..(1): basic amino acid
  misc_feature (2)..(4): Xaa can be any naturally occurring amino acid
  misc_feature (6)..(11): Xaa can be any naturally occurring amino acid
  PEPTIDE (12)..(12): C, S or T
XXXXCXXXXXXX

SEQ ID NO: 170 - protein, 4 aa, Artificial Sequence
  synthetic peptide
  misc_feature (2)..(2): Xaa can be any naturally occurring amino acid
  PEPTIDE (3)..(3): C, S or T
  PEPTIDE (4)..(4): basic amino acid
CXXX

SEQ ID NO: 171 - protein, 6 aa, Artificial Sequence
  synthetic peptide
  misc_feature (2)..(4): Xaa can be any naturally occurring amino acid
  PEPTIDE (5)..(5): C, S or T
  PEPTIDE (6)..(6): basic amino acid
CXXXXX

SEQ ID NO: 172 - protein, 7 aa, Artificial Sequence
  synthetic peptide
  misc_feature (2)..(5): Xaa can be any naturally occurring amino acid
  PEPTIDE (6)..(6): C, S or T
  PEPTIDE (7)..(7): basic amino acid
CXXXXXX

SEQ ID NO: 173 - protein, 8 aa, Artificial Sequence
  synthetic peptide
  misc_feature (2)..(6): Xaa can be any naturally occurring amino acid
  PEPTIDE (7)..(7): C, S or T
  PEPTIDE (8)..(8): basic amino acid
CXXXXXXX

SEQ ID NO: 174 - protein, 9 aa, Artificial Sequence
  synthetic peptide
  misc_feature (2)..(7): Xaa can be any naturally occurring amino acid
  PEPTIDE (8)..(8): C, S or T
  PEPTIDE (9)..(9): basic amino acid
CXXXXXXXX

SEQ ID NO: 175 - protein, 4 aa, Artificial Sequence
  synthetic peptide
  PEPTIDE (2)..(2): C, S or T
  misc_feature (3)..(3): Xaa can be any naturally occurring amino acid
  PEPTIDE (4)..(4): basic amino acid
CXXX

SEQ ID NO: 176 - protein, 5 aa, Artificial Sequence
  synthetic peptide
  misc_feature (2)..(2): Xaa can be any naturally occurring amino acid
  PEPTIDE (3)..(3): C, S or T
  misc_feature (4)..(4): Xaa can be any naturally occurring amino acid
  PEPTIDE (5)..(5): basic amino acid
CXXXX

SEQ ID NO: 177 - protein, 7 aa, Artificial Sequence
  synthetic peptide
  misc_feature (2)..(4): Xaa can be any naturally occurring amino acid
  PEPTIDE (5)..(5): C, S or T
  misc_feature (6)..(6): Xaa can be any naturally occurring amino acid
  PEPTIDE (7)..(7): basic amino acid
CXXXXXX

SEQ ID NO: 178 - protein, 8 aa, Artificial Sequence
  synthetic peptide
  misc_feature (2)..(5): Xaa can be any naturally occurring amino acid
  PEPTIDE (6)..(6): C, S or T
  misc_feature (7)..(7): Xaa can be any naturally occurring amino acid
  PEPTIDE (8)..(8): basic amino acid
CXXXXXXX

SEQ ID NO: 179 - protein, 9 aa, Artificial Sequence
  synthetic peptide
  misc_feature (2)..(6): Xaa can be any naturally occurring amino acid
  PEPTIDE (7)..(7): C, S or T
  misc_feature (8)..(8): Xaa can be any naturally occurring amino acid
  PEPTIDE (9)..(9): basic amino acid
CXXXXXXXX

SEQ ID NO: 180 - protein, 10 aa, Artificial Sequence
  synthetic peptide
  misc_feature (2)..(7): Xaa can be any naturally occurring amino acid
  PEPTIDE (8)..(8): C, S or T
  misc_feature (9)..(9): Xaa can be any naturally occurring amino acid
  PEPTIDE (10)..(10): basic amino acid
CXXXXXXXXX

SEQ ID NO: 181 - protein, 5 aa, Artificial Sequence
  synthetic peptide
  PEPTIDE (2)..(2): C, S or T
  misc_feature (3)..(4): Xaa can be any naturally occurring amino acid
  PEPTIDE (5)..(5): basic amino acid
CXXXX

SEQ ID NO: 182 - protein, 6 aa, Artificial Sequence
  synthetic peptide
  misc_feature (2)..(2): Xaa can be any naturally occurring amino acid
  PEPTIDE (3)..(3): C, S or T
  misc_feature (4)..(5): Xaa can be any naturally occurring amino acid
  PEPTIDE (6)..(6): basic amino acid
CXXXXX

SEQ ID NO: 183 - protein, 8 aa, Artificial Sequence
  synthetic peptide
  misc_feature (2)..(4): Xaa can be any naturally occurring amino acid
  PEPTIDE (5)..(5): C, S or T
  misc_feature (6)..(7): Xaa can be any naturally occurring amino acid
  PEPTIDE (8)..(8): basic amino acid
CXXXXXXX

SEQ ID NO: 184 - protein, 9 aa, Artificial Sequence
  synthetic peptide
  misc_feature (2)..(5): Xaa can be any naturally occurring amino acid
  PEPTIDE (6)..(6): C, S or T
  misc_feature (7)..(8): Xaa can be any naturally occurring amino acid
  PEPTIDE (9)..(9): basic amino acid
CXXXXXXXX

SEQ ID NO: 185 - protein, 10 aa, Artificial Sequence
  synthetic peptide
  misc_feature (2)..(6): Xaa can be any naturally occurring amino acid
  PEPTIDE (7)..(7): C, S or T
  misc_feature (8)..(9): Xaa can be any naturally occurring amino acid
  PEPTIDE (10)..(10): basic amino acid
CXXXXXXXXX

SEQ ID NO: 186 - protein, 11 aa, Artificial Sequence
  synthetic peptide
  misc_feature (2)..(7): Xaa can be any naturally occurring amino acid
  PEPTIDE (8)..(8): C, S or T
  misc_feature (9)..(10): Xaa can be any naturally occurring amino acid
  PEPTIDE (11)..(11): basic amino acid
CXXXXXXXXXX

SEQ ID NO: 187 - protein, 6 aa, Artificial Sequence
  synthetic peptide
  PEPTIDE (2)..(2): C, S or T
  misc_feature (3)..(5): Xaa can be any naturally occurring amino acid
  PEPTIDE (6)..(6): basic amino acid
CXXXXX

SEQ ID NO: 188 - protein, 7 aa, Artificial Sequence
  synthetic peptide
  misc_feature (2)..(2): Xaa can be any naturally occurring amino acid
  PEPTIDE (3)..(3): C, S or T
  misc_feature (4)..(6): Xaa can be any naturally occurring amino acid
  PEPTIDE (7)..(7): basic amino acid
CXXXXXX

SEQ ID NO: 189 - protein, 9 aa, Artificial Sequence
  synthetic peptide
  misc_feature (2)..(4): Xaa can be any naturally occurring amino acid
  PEPTIDE (5)..(5): C, S or T
  misc_feature (6)..(8): Xaa can be any naturally occurring amino acid
  PEPTIDE (9)..(9): basic amino acid
CXXXXXXXX

SEQ ID NO: 190 - protein, 10 aa, Artificial Sequence
  synthetic peptide
  misc_feature (2)..(5): Xaa can be any naturally occurring amino acid
  PEPTIDE (6)..(6): C, S or T
  misc_feature (7)..(9): Xaa can be any naturally occurring amino acid
  PEPTIDE (10)..(10): basic amino acid
CXXXXXXXXX

SEQ ID NO: 191 - protein, 11 aa, Artificial Sequence
  synthetic peptide
  misc_feature (2)..(6): Xaa can be any naturally occurring amino acid
  PEPTIDE (7)..(7): C, S or T
  misc_feature (8)..(10): Xaa can be any naturally occurring amino acid
  PEPTIDE (11)..(11): basic amino acid
CXXXXXXXXXX

SEQ ID NO: 192 - protein, 12 aa, Artificial Sequence
  synthetic peptide
  misc_feature (2)..(7): Xaa can be any naturally occurring amino acid
  PEPTIDE (8)..(8): C, S or T
  misc_feature (9)..(11): Xaa can be any naturally occurring amino acid
  PEPTIDE (12)..(12): basic amino acid
CXXXXXXXXXXX

SEQ ID NO: 193 - protein, 4 aa, Artificial Sequence
  synthetic peptide
  misc_feature (2)..(3): Xaa can be any naturally occurring amino acid
CXXC

SEQ ID NO: 194 - protein, 14 aa, Artificial Sequence
  synthetic peptide
  misc_feature (2)..(3): Xaa can be any naturally occurring amino acid
  misc_feature (5)..(5): Xaa can be any naturally occurring amino acid
  misc_feature (7)..(8): Xaa can be any naturally occurring amino acid
  misc_feature (10)..(10): Xaa can be any naturally occurring amino acid
  misc_feature (12)..(13): Xaa can be any naturally occurring amino acid
CXXCXCXXCXCXXC

SEQ ID NO: 195 - protein, 12 aa, Artificial Sequence
  synthetic peptide
  misc_feature (2)..(3): Xaa can be any naturally occurring amino acid
  misc_feature (6)..(7): Xaa can be any naturally occurring amino acid
  misc_feature (10)..(11): Xaa can be any naturally occurring amino acid
CXXCCXXCCXXC

SEQ ID NO: 196 - protein, 10 aa, Artificial Sequence
  synthetic peptide
  misc_feature (2)..(3): Xaa can be any naturally occurring amino acid
  misc_feature (5)..(6): Xaa can be any naturally occurring amino acid
  misc_feature (8)..(9): Xaa can be any naturally occurring amino acid
CXXCXXCXXC

SEQ ID NO: 197 - protein, 10 aa, Artificial Sequence
  synthetic peptide
  misc_feature (2)..(2): Xaa can be any naturally occurring amino acid
  misc_feature (5)..(5): Xaa can be any naturally occurring amino acid
  misc_feature (8)..(8): Xaa can be any naturally occurring amino acid
CXCCXCCXCC

SEQ ID NO: 198 - protein, 7 aa, Artificial Sequence
  synthetic peptide
  PEPTIDE (1)..(1): basic amino acid
  PEPTIDE (2)..(2): any amino acid and can be from 0 to 3 in length
  PEPTIDE (3)..(3): C, S, or T
  PEPTIDE (4)..(4): any amino acid and can be from 0 to 6 in length
  PEPTIDE (6)..(6): any amino acid and can be from 0 to 3 in length
  PEPTIDE (7)..(7): basic amino acid
XXXXCXX

SEQ ID NO: 199 - protein, 7 aa, Artificial Sequence
  synthetic peptide
  PEPTIDE (1)..(1): basic amino acid
  PEPTIDE (2)..(2): any amino acid and can be from 0 to 3 in length
  PEPTIDE (4)..(4): any amino acid and can be from 0 to 6 in length
  PEPTIDE (5)..(5): C, S, or T
  PEPTIDE (6)..(6): any amino acid and can be from 0 to 3 in length
  PEPTIDE (7)..(7): basic amino acid
XXCXXXX

SEQ ID NO: 200 - protein, 5 aa, Artificial Sequence
  synthetic peptide
  misc_feature (2)..(2): Xaa can be any naturally occurring amino acid
  PEPTIDE (3)..(3): K, H, or R
  misc_feature (4)..(4): Xaa can be any naturally occurring amino acid
CXXXC

SEQ ID NO: 201 - protein, 5 aa, Artificial Sequence
  synthetic peptide
  misc_feature (2)..(3): Xaa can be any naturally occurring amino acid
  PEPTIDE (4)..(4): K, H, or R
CXXXC

SEQ ID NO: 202 - protein, 5 aa, Artificial Sequence
  synthetic peptide
  PEPTIDE (2)..(2): K, H, or R
  misc_feature (3)..(4): Xaa can be any naturally occurring amino acid
CXXXC

SEQ ID NO: 203 - protein, 4 aa, Artificial Sequence
  synthetic peptide
  PEPTIDE (1)..(1): K, H, or R
  misc_feature (3)..(3): Xaa can be any naturally occurring amino acid
XCXC

SEQ ID NO: 204 - protein, 5 aa, Artificial Sequence
  synthetic peptide
  PEPTIDE (1)..(1): K, H, or R
  misc_feature (2)..(4): Xaa can be any naturally occurring amino acid
XXXXC

SEQ ID NO: 205 - protein, 4 aa, Artificial Sequence
  synthetic peptide
  misc_feature (2)..(2): Xaa can be any naturally occurring amino acid
  PEPTIDE (4)..(4): K, H, or R
CXCX

SEQ ID NO: 206 - protein, 6 aa, Artificial Sequence
  synthetic peptide
  misc_feature (2)..(4): Xaa can be any naturally occurring amino acid
  PEPTIDE (6)..(6): K, H, or R
CXXXCX

SEQ ID NO: 207 - protein, 4 aa, Artificial Sequence
  synthetic peptide
  PEPTIDE (1)..(1): K, H, or R
  PEPTIDE (4)..(4): K, H, or R
XCCX

SEQ ID NO: 208 - protein, 5 aa, Artificial Sequence
  synthetic peptide
  PEPTIDE (1)..(1): K, H, or R
  misc_feature (3)..(3): Xaa can be any naturally occurring amino acid
  PEPTIDE (5)..(5): K, H, or R
XCXCX

SEQ ID NO: 209 - protein, 7 aa, Artificial Sequence
  synthetic peptide
  PEPTIDE (1)..(1): K, H, or R
  misc_feature (3)..(5): Xaa can be any naturally occurring amino acid
  PEPTIDE (7)..(7): K, H, or R
XCXXXCX

SEQ ID NO: 210 - protein, 4 aa, Artificial Sequence
  synthetic peptide
  PEPTIDE (1)..(1): K, H, or R
  PEPTIDE (3)..(3): K, H, or R
XCXC

SEQ ID NO: 211 - protein, 4 aa, Artificial Sequence
  synthetic peptide
  PEPTIDE (2)..(2): K, H, or R
  PEPTIDE (4)..(4): K, H, or R
CXCX

SEQ ID NO: 212 - protein, 6 aa, Artificial Sequence
  synthetic peptide
  PEPTIDE (1)..(1): K, H, or R
  misc_feature (3)..(4): Xaa can be any naturally occurring amino acid
  PEPTIDE (5)..(5): K, H, or R
XCXXXC

SEQ ID NO: 213 - protein, 6 aa, Artificial Sequence
  synthetic peptide
  PEPTIDE (1)..(1): K, H, or R
  misc_feature (3)..(3): Xaa can be any naturally occurring amino acid
  PEPTIDE (4)..(4): K, H, or R
  misc_feature (5)..(5): Xaa can be any naturally occurring amino acid
XCXXXC

SEQ ID NO: 214 - protein, 5 aa, Artificial Sequence
  synthetic peptide
  PEPTIDE (1)..(1): K, H, or R
  misc_feature (2)..(2): Xaa can be any naturally occurring amino acid
  PEPTIDE (3)..(3): K, H, or R
  misc_feature (4)..(4): Xaa can be any naturally occurring amino acid
CXXXC

SEQ ID NO: 215 - protein, 6 aa, Artificial Sequence
  synthetic peptide
  misc_feature (2)..(3): Xaa can be any naturally occurring amino acid
  PEPTIDE (4)..(4): K, H, or R
  PEPTIDE (6)..(6): K, H, or R
CXXXCX

SEQ ID NO: 216 - protein, 6 aa, Artificial Sequence
  synthetic peptide
  misc_feature (2)..(2): Xaa can be any naturally occurring amino acid
  PEPTIDE (3)..(3): K, H, or R
  misc_feature (4)..(4): Xaa can be any naturally occurring amino acid
  PEPTIDE (6)..(6): K, H, or R
CXXXCX

SEQ ID NO: 217 - protein, 6 aa, Artificial Sequence
  synthetic peptide
  PEPTIDE (2)..(2): K, H, or R
  misc_feature (3)..(4): Xaa can be any naturally occurring amino acid
  PEPTIDE (6)..(6): K, H, or R
CXXXCX

SEQ ID NO: 218 - protein, 4 aa, Artificial Sequence
  synthetic peptide
CPYC

SEQ ID NO: 219 - protein, 5 aa, Artificial Sequence
  synthetic peptide
KCPYC

SEQ ID NO: 220 - protein, 4 aa, Artificial Sequence
  synthetic peptide
  misc_feature (3)..(3): Xaa can be any naturally occurring amino acid
KCXC

SEQ ID NO: 221 - protein, 6 aa, Artificial Sequence
  synthetic peptide
  misc_feature (3)..(5): Xaa can be any naturally occurring amino acid
KCXXXC

SEQ ID NO: 222 - protein, 6 aa, Artificial Sequence
  synthetic peptide
KCKPYC

(19) United States
(12) Patent Application Publication
(10) Pub. No.: US 2022/0288179 A1
(43) Pub. Date: Sep. 15, 2022

(54) IMMUNOGENIC PEPTIDES WITH NEW OXIDOREDUCTASE MOTIFS

(71) Applicant: IMCYSE SA, Liege (BE)

(72) Inventor: Milos ERAK, Brussels (BE)

(21) Appl. No.: 17/292,888

(22) PCT Filed: Nov. 12, 2019
(86) PCT No.: PCT/EP2019/080925

§ 371 Date: May 11, 2021

(30) Foreign Application Priority Data
Nov. 12, 2018 (EP) 18205611.9
Nov. 12, 2018 (EP) 18205615.0

(51) Int. Cl. A61K 39/00; C12N 5/0783; C12N 9/02; C12N 15/62

(52) U.S. Cl. CPC A61K 39/0011; A61K 39/0008; C12N 5/0636; C12N 9/0004; C12N 15/62; A61K 2039/5158; A61K 2039/6031; C12N 2501/2302; C12N 2501/998

(57) ABSTRACT

The invention relates to immunogenic peptides comprising T-cell epitopes and oxidoreductase motifs with increased activity, and their use in regulating the immune response in subjects.

### CROSS-REFERENCE TO RELATED APPLICATIONS

This application is the U.S. National Phase of International Application No. PCT/EP2019/080925, filed Nov. 12, 2019, which claims the benefit of European Patent Application No. 18205611.9, filed Nov. 12, 2018, and European Patent Application No. 18205615.0, filed Nov. 12, 2018, and which is incorporated herein by reference in its entirety.

### REFERENCE TO SEQUENCE LISTING SUBMITTED ELECTRONICALLY

The content of the electronically submitted sequence listing (Name: 2752_0134 Sequence_Listing.txt; Size: 87.8 kilobytes; and Date of Creation: May 3, 2021) filed with the application is incorporated herein by reference in its entirety.

### BACKGROUND OF THE INVENTION

Several strategies have been described to prevent the generation of an unwanted immune response against an antigen. WO2008/017517 describes a new strategy using peptides comprising an MHC class II antigen of a given antigenic protein and an oxidoreductase motif. These peptides convert CD4+ T cells into a cell type with cytolytic properties called cytolytic CD4+ T cells. These cells are capable to kill via triggering apoptosis those antigen presenting cells (APC), which present the antigen from which the peptide is derived. WO2008/017517 demonstrates this concept for allergies and auto-immune diseases such as type I diabetes. Herein insulin can act as an auto-antigen.

WO2009101207 and Carlier et al. (2012) Plos one 7,10 e45366 further describe the antigen specific cytolytic cells in more detail.

WO2009101206 describes the use of peptides with an oxidoreductase motif and an MCH class II epitope of a soluble allo-antigen to prevent an immune response against such antigen when used in replacement therapies (e.g. unwanted immune response against injected insulin in diabetes patents).

WO2016059236 discloses further modified peptides wherein an additional Histidine is present in the proximity of the oxidoreductase motif.

In the design of a peptide against type I diabetes, many factors can be taken into account, such as the type of the auto-antigen (insulin, GAD 65, . . . ), a specific domain and epitope of the auto-antigen, the oxidoreductase motif, the length and amino-acid acid sequence between the oxidoreductase motif and the epitope sequence.

In addition to the peptides comprising an MHC class II epitope of an allergen or antigen, WO2012069568A2 further disclosed the possibility of using NKT cell epitopes, binding the CD1d receptor and resulting in activation of cytolytic antigen-specific NKT cells, which have been shown to eliminate, in an antigen-specific manner, APC presenting said specific antigen. Both strategies are building upon the use of oxidoreductase motifs of the [CST]X2C or CX2[CST] type. In order to improve the efficacy of a treatment using such immunogenic peptides, the search for more active peptides and/or more potent oxidoreductase motifs continues.

### SUMMARY OF THE INVENTION

The present invention provides novel immunogenic peptides comprising a T-cell epitope of an antigen and an oxidoreductase motif. After conducting extensive experiments the inventors have identified a new type of oxidoreductase motifs with different activities when compared to the traditionally used CXX[CST] or [CST]XXC oxidoreductase motifs. When combining these with an (additional) basic (charged) amino acid residue before, inside or after said new motifs, the oxidoreductase activity was improved. By doing this, the inventors found that in many cases, the oxidoreductase activity is changed when using specific combinations of new motifs and/or basic amino acids as claimed. This implies that the choice of a certain basic amino acid in the motif is not arbitrary but leads to an improved effect of the motif. More in particular, the inventors have shown that the use of basic amino acids K (lysine) or R (arginine) outperform the use of H (histidine). In some specific positions, also the K and R residues outperform each other and combinations of multiple basic amino acid residues in the motif seem to further increase these effects. These effects are displayed in the Figures and explained in the Examples section. Also the effect on the cellular level in model systems has been tested and confirms the improved activity of the immunogenic peptides of the invention.

The present invention relates to the following aspects:

Aspect 1: An immunogenic peptide, said immunogenic peptide comprising:

a) an oxidoreductase motif,

b) a T-cell epitope of an antigenic protein,

c) a linker between a) and b) of between 0 and 7 amino acids,

wherein said oxidoreductase motif is selected from the group comprising: [CST]XₙC or CXₙ[CST],

wherein X is any amino acid, and

wherein n is an integer selected from the group comprising: 0, 1, 3, 4, 5 or 6, with the proviso that when n is 0, said oxidoreductase motif is not part of a repeat of the standard CXXC oxidoreductase motifs such as repeats of said motif which can be spaced from each other by one or more amino acids (e.g. CXXC X CXXC X CXXC), as repeats which are adjacent to each other (CXXC CXXC CXXC) or as repeats which overlap with each other CXXCXXCXXC or CXCCXCCXCC).

Aspect 2: The immunogenic peptide according to aspect 1, wherein at least one X in the motif is a basic amino acid.

Aspect 3: The immunogenic peptide according to aspect 2, wherein said basic amino acid is selected from the group comprising: K, H, R or a non-natural basic amino acid.

Aspect 4: The immunogenic peptide according to aspect 2 or 3, wherein said basic amino acid is K or L-ornithine.

Aspect 5: The immunogenic peptide according to any one of aspects 1 to 4, wherein said T cell epitope of an antigenic protein is an NKT cell epitope or an MHC class II T cell epitope.

Aspect 6: The immunogenic peptide according to any one of aspects 1 to 5, wherein said epitope has a length of between 7 and 25 amino acids.

Aspect 7: The immunogenic peptide according to any one of aspects 1 to 6, having a length of between 9 and 50 amino acids.

Aspect 8: The immunogenic peptide according to any one of aspects 1 to 7, wherein said antigenic protein is an auto-antigen, a soluble allofactor, an alloantigen shed by the graft, an antigen of an intracellular pathogen, an antigen of a viral vector used for gene therapy or gene vaccination, a tumor-associated antigen or an allergen.

Aspect 9: The immunogenic peptide according to any one of aspects 1 to 8, for use in medicine.

Aspect 10: The immunogenic peptide according to any one of aspects 1 to 9, for use in treating and/or prevention of an autoimmune disease, an infection with an intracellular pathogen, a tumor, an allograft rejection, or an immune response to a soluble allofactors, to an allergen exposure or to a viral vector used for gene therapy or gene vaccination.

Aspect 11: The immunogenic peptide according to any one of aspects 1 to 10, wherein at least one X in the motif is P or Y.

Aspect 12: The immunogenic peptide according to any one of aspects 1 to 11, wherein the linker is of between 0 and 4 amino acids.

Aspect 13: The immunogenic peptide according to any one of aspects 1 to 12, wherein the oxidoreductase motif is located N-terminally from the epitope or C-terminally from the epitope.

Aspect 14: The immunogenic peptide according to any one of aspects 1 to 13, wherein said oxidoreductase motif does not naturally occur within a region of 11 amino acids N-terminally or C-terminally of the T-cell epitope in said antigenic protein.

Aspect 15: The immunogenic peptide according to any one of aspects 1 to 14, wherein the T-cell epitope does not naturally comprise said oxidoreductase motif.

Aspect 16: The immunogenic peptide according to any one of aspects 1 to 15, wherein said oxidoreductase motif is selected from the group comprising: ZBₘ[CST]XₙC, [CST]XₙCBₘZ, ZBₘCXₙ[CST], or CXₙ[CST]BₘZ

wherein X or B is any amino acid,

wherein n is an integer selected from the group comprising: 0, 1, 3, 4, 5 or 6,

wherein m is an integer between 0 and 3,

wherein Z is a basic amino acid, preferably selected from the group comprising: K, H, R or a non-natural basic amino acid, preferably wherein each of Bₘ is K, H, or R, more preferably H.

Aspect 17: The immunogenic peptide according to aspect 16, wherein said basic amino acid is selected from the group comprising: K, H, R or a non-natural basic amino acid.

Aspect 18: The immunogenic peptide according to aspects 16 or 17, wherein said basic amino acid is K or L-ornithine.

Aspect 19: The immunogenic peptide according to any one of aspects 1 to 15, wherein said oxidoreductase motif is selected from the group comprising:

Z¹-Bₗ-[CST]-Xₙ-C,

Z¹-Bₗ-C-Xₙ-[CST],

[CST]-Xₙ-C-Bₘ-Z²,

C-Xₙ-[CST]-Bₘ-Z²,

Z¹-Bₗ-[CST]-Xₙ-C-Bₘ-Z² or

Z¹-Bₗ-C-Xₙ-[CST]-Bₘ-Z²,

wherein each of X, Bₗ and Bₘ is any amino acid, preferably wherein at least one X in the motif is a basic amino acid preferably selected from the group comprising: K, H, R or a non-natural basic amino acid, preferably wherein each of Bₗ and/or Bₘ is K, H, or R, more preferably H,

wherein n is an integer selected from the group comprising: 0, 1, 3, 4, 5 or 6,

wherein l and m are an integer selected from the group comprising 0 to 3,

wherein Z¹ and Z² are basic amino acids, preferably selected from the group comprising: K, H, R or a non-natural basic amino acid.

Aspect 20: The immunogenic peptide according to aspect 19, wherein n is an integer selected from 0, 1 or 3; and wherein said at least one basic amino acid is K or R.

Aspect 21: A method for preparing an immunogenic peptide according to any one of aspects 1 to 20, comprising the steps of:

(a) providing a peptide sequence consisting of a T-cell epitope of said antigenic protein, and

(b) linking to said peptide sequence said oxidoreductase motif, such that said motif and said epitope are either adjacent to each other or separated by a linker of between 0 and 7 amino acids.

Aspect 22: A method for obtaining a population of antigen-specific cytolytic CD4+ T cells, against APC presenting said antigen, the method comprising the steps of:
providing peripheral blood cells,
contacting said cells with an immunogenic peptide according to any one of aspects 1 to 20; and
expanding said cells in the presence of IL-2.

Aspect 23: A method for obtaining a population of antigen-specific NKT cells, the method comprising the steps of:
providing peripheral blood cells,
contacting said cells with an immunogenic peptide according to any one of aspects 1 to 20; and
expanding said cells in the presence of IL-2.

Aspect 24: A method for obtaining a population of antigen-specific cytolytic CD4+ T cells, against APC presenting said antigen, the method comprising the steps of:
providing an immunogenic peptide according to any one of aspects 1 to 20,
administering said peptide to a subject, and
obtaining said population of antigen-specific cytolytic CD4+ T cells from said subject.

Aspect 25: A method for obtaining a population of antigen-specific NKT cells, the method comprising the steps of:
providing an immunogenic peptide according to any of aspects 1 to 20;
administering said peptide to a subject, and
obtaining said population of antigen-specific NKT cells from said subject.

Aspect 26: The population of antigen-specific cytolytic CD4+ T cells or NKT cells obtainable by the method of any one of aspects 22 to 25 for use in medicine, more particularly for use in the treatment and/or prevention of an autoimmune disease, an infection with an intracellular pathogen, a tumor, an allograft rejection, or an immune response to a soluble allofactors, to an allergen exposure or to a viral vector used for gene therapy or gene vaccination.

Aspect 27: A method of treating and/or preventing an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of administering the immunogenic peptide according to anyone of aspects 1 to 20, more particularly comprising:

a) an oxidoreductase motif,

b) a T-cell epitope of an antigenic protein,

c) a linker between a) and b) of between 0 and 7 amino acids,

wherein said oxidoreductase motif is selected from the group comprising: [CST]XₙC or CXₙ[CST],

wherein X is any amino acid, and

wherein n is an integer selected from the group comprising: 0, 1, 3, 4, 5 or 6;

or the cell population according to claim 24 to said individual.

Aspect 28: A method of treating or preventing an autoimmune disease, an infection with an intracellular pathogen, a tumor, an allograft rejection, or an immune response to a soluble allofactors, to an allergen exposure or to a viral vector used for gene therapy or gene vaccination in an individual, comprising the steps of:
providing peripheral blood cells of said individual,
contacting said cells with an antigenic peptide according to any one of aspects 1 to 20, more particularly comprising:

a) an oxidoreductase motif,

b) a T-cell epitope of an antigenic protein,

c) a linker between a) and b) of between 0 and 7 amino acids,

wherein said oxidoreductase motif is selected from the group comprising: [CST]XₙC or CXₙ[CST],

wherein X is any amino acid, and

wherein n is an integer selected from the group comprising: 0, 1, 3, 4, 5 or 6;
expanding said cells, and
administering said expanded cells to said individual.

Particularly preferred examples of oxidoreductase motifs to be read into any one of the aspects or embodiments disclosed herein are: C[KHR]C, CX[KHR]XC, CXX[KHR]C, C[KHR]XXC, [KHR]CC, [KHR]CXC, [KHR]XXXC CC[KHR], CXC[KHR], CXXXC[KHR], [KHR]CC[KHR], [KHR]CXC[KHR], [KHR]CXXXC[KHR], [KHR]C[KHR]C, C[KHR]C[KHR], [KHR]CXX[KHR]C, [KHR]CX[KHR]XC, [KHR]C[KHR]XXC, CXX[KHR]C[KHR], CX[KHR]XC[KHR], C[KHR]XXC[KHR], and the like.

In a preferred embodiment of any one of said aspects, the linker comprises at least 1 amino acid, at least 2 amino acids, at least 3 amino acids, or at least 4 amino acids. Preferably, said linker comprises between 1 and 7 amino acids, such as between 2 and 7 amino acids, between 3 and 7 amino acids, or between 4 and 7 amino acids.

In another preferred embodiment of any one of said aspects, the T-cell epitope does not comprise a basic amino acid at its N-terminal end, i.e. immediately adjacent to the linker or oxidoreductase motif, more particularly in case the linker is absent or only comprises 1 or 2 amino acids. More preferably, in all aspects, the T-cell epitope does not comprise a basic amino acid at its N-terminal end, i.e. immediately adjacent to the linker or oxidoreductase motif, more particularly in case the linker is absent or only comprises 1 or 2 amino acids.

In a further embodiment of any one of said aspects, the T-cell epitope does not comprise a basic amino acid in position 1, 2 and/or 3 counted from its N-terminal end, i.e. immediately adjacent to the linker or oxidoreductase motif, more particularly in case the linker is absent or only comprises 1 or 2 amino acids.

In a further embodiment of any one of said aspects, either one of X, or B can be a basic amino acid. In another embodiment, either one of X or B is any amino acid except for C, S, or T. In yet a further embodiment, either one of X or B is any amino acid except for a basic amino acid.

In the above embodiments the redox motif is at the N terminal side of the epitope. In an alternative set of embodiments the peptides have the redox motif at the C terminal side of the epitope.

The peptides of the present invention have the advantage that cytolytic CD4+ T cells which have been generated using these peptides have an increased IFN-gamma and sFasL production compared to prior art peptides. Also Granzyme B production in said CD4+ T cells is believed to be increased.

The increased expression levels of these markers are indications of a greater capacity of the peptides of the present invention to generate cytolytic CD4+ T cells compared to the prior art peptides.

### BRIEF DESCRIPTION OF THE FIGURES

FIG. 1: represents the kinetics of the oxidoreductase activities of immunogenic peptides comprising a KCC motif and a tetanus toxin T cell epitope. The initial velocities of the redox activities are followed for 10 minutes. The same peptide but with the CPYC or KCPYC sequence is used as the control peptide. See table 1 for details.

FIG. 2: represents the kinetics of the oxidoreductase activities of immunogenic peptides comprising the KCxC motif and a tetanus toxin T cell epitope. The initial velocities of the redox activities are followed for 10 minutes. The same peptide but with the CPYC or KCPYC sequence is used as the control peptide. See table 2 for details.

FIG. 3a: represents the kinetics of the oxidoreductase activities of immunogenic peptides comprising the KCxxxC motif and a tetanus toxin T cell epitope, wherein one x is the basic amino acid K. The initial velocities of the redox activities are followed for 10 minutes. The same peptide but with the CPYC or KCPYC sequence is used as the control peptide. See table 3a for details.

FIG. 3b: represents the kinetics of the oxidoreductase activities of immunogenic peptides comprising the KCxxxC motif and a tetanus toxin T cell epitope, wherein one x is the basic amino acid R. The initial velocities of the redox activities are followed for 10 minutes. The same peptide but with the CPYC or KCPYC sequence is used as the control peptide. See table 3b for details.

FIG. 3c: represents the kinetics of the oxidoreductase activities of immunogenic peptides comprising the KCxxxC motif and a tetanus toxin T cell epitope, wherein one x is the basic amino acid H. The initial velocities of the redox activities are followed for 10 minutes. The same peptide but with the CPYC or KCPYC sequence is used as the control peptide. See table 3c for details.

FIG. 3d: represents the kinetics of the oxidoreductase activities of immunogenic peptides comprising the KCxxxC motif and a tetanus toxin T cell epitope, wherein the third x is the amino acid A. The initial velocities of the redox activities are followed for 10 minutes. The same peptide but with the CPYC or KCPYC sequence is used as the control peptide. See table 3d for details.

### DETAILED DESCRIPTION OF THE INVENTION

The present invention will be described with respect to particular embodiments but the invention is not limited thereto but only by the claims. Any reference signs in the claims shall not be construed as limiting the scope. The following terms or definitions are provided solely to aid in the understanding of the invention. Unless specifically defined herein, all terms used herein have the same meaning as they would have to one skilled in the art of the present invention. The definitions provided herein should not be construed to have a scope less than the one understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and the include both singular and plural referents unless the context clearly dictates otherwise. The term “any” when used in relation to aspects, claims or embodiments as used herein refers to any single one (i.e. anyone) as well as to all combinations of said aspects, claims or embodiments referred to.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. Said terms also encompass the embodiments “consisting essentially of” and “consisting of”.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

As used herein, the term “for use” as used in “composition for use in treatment of a disease” shall disclose also the corresponding method of treatment and the corresponding use of a preparation for the manufacture of a medicament for the treatment of a disease”.

The term “peptide” as used herein refers to a molecule comprising an amino acid sequence of between 12 and 200 amino acids, connected by peptide bonds, but which can comprise non-amino acid structures.

The term “immunogenic peptide” as used herein refers to a peptide that is immunogenic, i.e. that comprises a T-cell epitope capable of eliciting an immune response.

Peptides according to the invention can contain any of the conventional 20 amino acids or modified versions thereof, or can contain non-naturally occurring amino-acids incorporated by chemical peptide synthesis or by chemical or enzymatic modification.

The term “antigen” as used herein refers to a structure of a macromolecule, typically a protein (with or without polysaccharides) or made of proteic composition comprising one or more hapten(s) and comprising T or NKT cell epitopes.

The term “antigenic protein” as used herein refers to a protein comprising one or more T or NKT cell epitopes. An auto-antigen or auto-antigenic protein as used herein refers to a human or animal protein or fragment thereof present in the body, which elicits an immune response within the same human or animal body.

The term “food or pharmaceutical antigenic protein” refers to an antigenic protein present in a food or pharmaceutical product, such as in a vaccine.

The term “epitope” refers to one or several portions (which may define a conformational epitope) of an antigenic protein which is/are specifically recognised and bound by an antibody or a portion thereof (Fab′, Fab2′, etc.) or a receptor presented at the cell surface of a B-, or T-, or NKT cell, and which is able, by said binding, to induce an immune response.

The term “T cell epitope” in the context of the present invention refers to a dominant, sub-dominant or minor T cell epitope, i.e. a part of an antigenic protein that is specifically recognised and bound by a receptor at the cell surface of a T lymphocyte. Whether an epitope is dominant, sub-dominant or minor depends on the immune reaction elicited against the epitope. Dominance depends on the frequency at which such epitopes are recognised by T cells and able to activate them, among all the possible T cell epitopes of a protein.

The T cell epitope is an epitope recognised by MHC class II molecules, which consists of a sequence of +/−9 amino acids which fit in the groove of the MHC II molecule. Within a peptide sequence representing a T cell epitope, the amino acids in the epitope are numbered P1 to P9, amino acids N-terminal of the epitope are numbered P-1, P-2 and so on, amino acids C terminal of the epitope are numbered P+1, P+2 and so on. Peptides recognised by MHC class II molecules and not by MHC class I molecules are referred to as MHC class II restricted T cell epitopes.

The identification and selection of a T-cell epitope from antigenic proteins is known to a person skilled in the art.

To identify an epitope suitable in the context of the present invention, isolated peptide sequences of an antigenic protein are tested by, for example, T cell biology techniques, to determine whether the peptide sequences elicit a T cell response. Those peptide sequences found to elicit a T cell response are defined as having T cell stimulating activity.

Human T cell stimulating activity can further be tested by culturing T cells obtained from e.g. an individual having T1 D, with a peptide/epitope derived from the auto-antigen involved in T1D and determining whether proliferation of T cells occurs in response to the peptide/epitope as measured, e.g., by cellular uptake of tritiated thymidine. Stimulation indices for responses by T cells to peptides/epitopes can be calculated as the maximum CPM in response to a peptide/epitope divided by the control CPM. A T cell stimulation index (S.I.) equal to or greater than two times the background level is considered “positive.” Positive results are used to calculate the mean stimulation index for each peptide/epitope for the group of peptides/epitopes tested.

Non-natural (or modified) T-cell epitopes can further optionally be tested on their binding affinity to MHC class II molecules. This can be performed in different ways. For instance, soluble HLA class II molecules are obtained by lysis of cells homozygous for a given class II molecule. The latter is purified by affinity chromatography. Soluble class II molecules are incubated with a biotin-labelled reference peptide produced according to its strong binding affinity for that class II molecule. Peptides to be assessed for class II binding are then incubated at different concentrations and their capacity to displace the reference peptide from its class II binding is calculated by addition of neutravidin.

In order to determine optimal T cell epitopes by, for example, fine mapping techniques, a peptide having T cell stimulating activity and thus comprising at least one T cell epitope as determined by T cell biology techniques is modified by addition or deletion of amino acid residues at either the amino- or carboxyterminus of the peptide and tested to determine a change in T cell reactivity to the modified peptide. If two or more peptides which share an area of overlap in the native protein sequence are found to have human T cell stimulating activity, as determined by T cell biology techniques, additional peptides can be produced comprising all or a portion of such peptides and these additional peptides can be tested by a similar procedure. Following this technique, peptides are selected and produced recombinantly or synthetically. T cell epitopes or peptides are selected based on various factors, including the strength of the T cell response to the peptide/epitope (e.g., stimulation index) and the frequency of the T cell response to the peptide in a population of individuals.

Additionally and/or alternatively, one or more in vitro algorithms can be used to identify a T cell epitope sequence within an antigenic protein. Suitable algorithms include, but are not limited to those described in Zhang et al. (2005) Nucleic Acids Res 33, W180-W183 (PREDBALB); Salomon & Flower (2006) BMC Bioinformatics 7, 501 (MHCBN); Schuler et al. (2007) Methods Mol. Biol.409, 75-93 (SYFPEITHI); Donnes & Kohlbacher (2006) Nucleic Acids Res. 34, W194-W197 (SVMHC); Kolaskar & Tongaonkar (1990) FEBS Lett. 276, 172-174, Guan et al. (2003) Appl. Bioinformatics 2, 63-66 (MHCPred) and Singh and Raghava (2001) Bioinformatics 17, 1236-1237 (Propred). More particularly, such algorithms allow the prediction within an antigenic protein of one or more octa- or nonapeptide sequences which will fit into the groove of an MHC II molecule and this for different HLA types.

The term “MHC” refers to “major histocompatibility antigen”. In humans, the MHC genes are known as HLA (“human leukocyte antigen”) genes. Although there is no consistently followed convention, some literature uses HLA to refer to HLA protein molecules, and MHC to refer to the genes encoding the HLA proteins. As such the terms “MHC” and “HLA” are equivalents when used herein. The HLA system in man has its equivalent in the mouse, i.e., the H2 system. The most intensely-studied HLA genes are the nine so-called classical MHC genes:HLA-A, HLA-B, HLA-C, HLA-DPA1, HLA-DPB1, HLA-DQA1, HLAs DQB1, HLA-DRA, and HLA-DRB1. In humans, the MHC is divided into three regions:Class I, II, and III. The A, B, and C genes belong to MHC class I, whereas the six D genes belong to class II. MHC class I molecules are made of a single polymorphic chain containing 3 domains (alpha 1, 2 and 3), which associates with beta 2 microglobulin at cell surface. Class II molecules are made of 2 polymorphic chains, each containing 2 chains (alpha 1 and 2, and beta 1 and 2).

Class I MHC molecules are expressed on virtually all nucleated cells.

Peptide fragments presented in the context of class I MHC molecules are recognised by CD8+ T lymphocytes (cytolytic T lymphocytes or CTLs). CD8+ T lymphocytes frequently mature into cytolytic effectors which can lyse cells bearing the stimulating antigen. Class II MHC molecules are expressed primarily on activated lymphocytes and antigen-presenting cells. CD4+ T lymphocytes (helper T lymphocytes or Th) are activated with recognition of a unique peptide fragment presented by a class II MHC molecule, usually found on an antigen-presenting cell like a macrophage or dendritic cell. CD4+ T lymphocytes proliferate and secrete cytokines such as IL-2, IFN-gamma and IL-4 that support antibody-mediated and cell mediated responses.

Functional HLAs are characterised by a deep binding groove to which endogenous as well as foreign, potentially antigenic peptides bind. The groove is further characterised by a well-defined shape and physico-chemical properties. HLA class I binding sites are closed, in that the peptide termini are pinned down into the ends of the groove. They are also involved in a network of hydrogen bonds with conserved HLA residues. In view of these restraints, the length of bound peptides is limited to 8, 9 or 10 residues. However, it has been demonstrated that peptides of up to 12 amino acid residues are also capable of binding HLA class I. Comparison of the structures of different HLA complexes confirmed a general mode of binding wherein peptides adopt a relatively linear, extended conformation, or can involve central residues to bulge out of the groove.

In contrast to HLA class I binding sites, class II sites are open at both ends. This allows peptides to extend from the actual region of binding, thereby “hanging out” at both ends. Class II HLAs can therefore bind peptide ligands of variable length, ranging from 9 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N-and C-terminal residues of the peptide but distributed over the whole chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class I. There is much more cross reactivity of peptides among different HLA class II allotypes. The sequence of the +/−9 amino acids (i.e. 8, 9 or 10) of an MHC class II T cell epitope that fit in the groove of the MHC II molecule are usually numbered P1 to P9. Additional amino acids N-terminal of the epitope are numbered P-1, P-2 and so on, amino acids C-terminal of the epitope are numbered P+1, P+2 and so on.

The term “NKT cell epitope” refers to a part of an antigenic protein that is specifically recognized and bound by a receptor at the cell surface of an NKT cell. In particular, a NKT cell epitope is an epitope bound by CD1d molecules. The NKT cell epitope has a general motif [FWYHT]-X(2)-[VILM]-X(2)-[FWYHT]. Alternative versions of this general motif have at position 1 and/or position 7 the alternatives [FWYH], thus [FWYH]-X(2)-[VILM]-X(2)-[FWYH].

Alternative versions of this general motif have at position 1 and/or position 7 the alternatives [FWYT], [FWYT]-X(2)-[VILM]-X(2)-[FWYT]. Alternative versions of this general motif have at position 1 and/or position 7 the alternatives [FWY], [FWY]-X(2)-[VILM]-X(2)-[FWY].

Regardless of the amino acids at position 1 and/or 7, alternative versions of the general motif have at position 4 the alternatives [ILM], e.g. [FWYH]-X(2)-[ILM]-X(2)-[FWYH] or [FWYHT]-X(2)-[ILM]-X(2)-[FWYHT] or [FWY]-X(2)-[ILM]-X(2)-[FWY].

A CD1d binding motif in a protein can be identified by scanning a sequence for the above sequence motifs, either by hand, either by using an algorithm such as ScanProsite De Castro E. et al. (2006) Nucleic Acids Res. 34(Web Server issue):W362-W365.

“Natural killer T” or “NKT” cells constitute a distinct subset of non-conventional T lymphocytes that recognize antigens presented by the non-classical MHC complex molecule CD1d. Two subsets of NKT cells are presently described. Type I NKT cells, also called invariant NKT cells (iNKT), are the most abundant. They are characterized by the presence of an alpha- beta T cell receptor (TCR) made of an invariant alpha chain, Valphal4 in the mouse and Valpha24 in humans. This alpha chain is associated to a variable though limited number of beta chains. Type 2 NKT cells have an alpha-beta TCR but with a polymorphic alpha chain. However, it is apparent that other subsets of NKT cells exist, the phenotype of which is still incompletely defined, but which share the characteristics of being activated by glycolipids presented in the context of the CD1d molecule. NKT cells typically express a combination of natural killer (NK) cell receptor, including NKG2D and NK1.1. NKT cells are part of the innate immune system, which can be distinguished from the adaptive immune system by the fact that they do not require expansion before acquiring full effector capacity. Most of their mediators are preformed and do not require transcription. NKT cells have been shown to be major participants in the immune response against intracellular pathogens and tumor rejection. Their role in the control of autoimmune diseases and of transplantation rejection is also advocated.

The recognition unit, the CD1d molecule, has a structure closely resembling that of the MHC class I molecule, including the presence of beta-2 microglobulin. It is characterized by a deep cleft bordered by two alpha chains and containing highly hydrophobic residues, which accepts lipid chains. The cleft is open at both extremities, allowing it to accommodate longer chains. The canonical ligand for CD1d is the synthetic alpha galactosylceramide (alpha GalCer). However, many natural alternative ligands have been described, including glyco- and phospholipids, the natural lipid sulfatide found in myelin, microbial phosphoinositol mannoside and alpha-glucuronosylceramide. The present consensus in the art (Matsuda et al (2008), Curr. Opinion Immunol., 20 358-368; Godfrey et al (2010), Nature rev. Immunol 11, 197-206) is still that CD1d binds only ligands containing lipid chains, or in general a common structure made of a lipid tail which is buried into CD1d and a sugar residue head group that protrudes out of CD1d.

The term “homologue” as used herein with reference to the epitopes used in the context of the invention, refers to molecules having at least 50%, at least 70%, at least 80%, at least 90%, at least 95% or at least 98% amino acid sequence identity with the naturally occurring epitope, thereby maintaining the ability of the epitope to bind an antibody or cell surface receptor of a B and/or T cell. Particular homologues of an epitope correspond to the natural epitope modified in at most three, more particularly in at most 2, most particularly in one amino acid.

The term “derivative” as used herein with reference to the peptides of the invention refers to molecules which contain at least the peptide active portion (i.e. the redox motif and the MHC class II epitope capable of eliciting cytolytic CD4+ T cell activity) and, in addition thereto comprises a complementary portion which can have different purposes such as stabilising the peptides or altering the pharmacokinetic or pharmacodynamic properties of the peptide.

The term “sequence identity” of two sequences as used herein relates to the number of positions with identical nucleotides or amino acids divided by the number of nucleotides or amino acids in the shorter of the sequences, when the two sequences are aligned. In particular, the sequence identity is from 70% to 80%, from 81% to 85%, from 86% to 90%, from 91% to 95%, from 96% to 100%, or 100%.

The terms “peptide-encoding polynucleotide (or nucleic acid)” and “polynucleotide (or nucleic acid) encoding peptide” as used herein refer to a nucleotide sequence, which, when expressed in an appropriate environment, results in the generation of the relevant peptide sequence or a derivative or homologue thereof. Such polynucleotides or nucleic acids include the normal sequences encoding the peptide, as well as derivatives and fragments of these nucleic acids capable of expressing a peptide with the required activity. The nucleic acid encoding a peptide according to the invention or fragment thereof is a sequence encoding the peptide or fragment thereof originating from a mammal or corresponding to a mammalian, most particularly a human peptide fragment.

The term “oxidoreductase motif”, “thiol-oxidoreductase motif”, “thioreductase motif”, “thioredox motif” or “redox motif” are used herein as synonymous terms and refers to motifs involved in the transfer of electrons from one molecule (the reductant, also called the hydrogen or electron donor) to another (the oxidant, also called the hydrogen or electron acceptor). In particular, the term “oxidoreductase motif” can refer to the known [CST]XXC or CXX[CST] motifs, but in particularly refers to to the sequence motif [CST]XₙC or CXₙ[CST], wherein n is an integer selected from the group comprising: 0, 1, 3, 4, 5 or 6, and in which C stands for cysteine, S for serine, T for threonine and X for any amino acid.

The term “basic amino acid” refers to any amino acid that acts like a Bronsted-Lowry and Lewis base, and includes natural basic amino acids such as Arginine (R), Lysine (K) or Histidine (H), or non-natural basic amino acids, such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)-OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

The term “immune disorders” or “immune diseases” refers to diseases wherein a reaction of the immune system is responsible for or sustains a malfunction or non-physiological situation in an organism. Included in immune disorders are, inter alia, allergic disorders and autoimmune diseases.

The terms “allergic diseases” or “allergic disorders” as used herein refer to diseases characterised by hypersensitivity reactions of the immune system to specific substances called allergens (such as pollen, stings, drugs, or food). Allergy is the ensemble of signs and symptoms observed whenever an atopic individual patient encounters an allergen to which he has been sensitised, which may result in the development of various diseases, in particular respiratory diseases and symptoms such as bronchial asthma. Various types of classifications exist and mostly allergic disorders have different names depending upon where in the mammalian body it occurs. “Hypersensitivity” is an undesirable (damaging, discomfort-producing and sometimes fatal) reaction produced in an individual upon exposure to an antigen to which it has become sensitised; “immediate hypersensitivity” depends of the production of IgE antibodies and is therefore equivalent to allergy.

The terms “autoimmune disease” or “autoimmune disorder” refer to diseases that result from an aberrant immune response of an organism against its own cells and tissues due to a failure of the organism to recognise its own constituent parts (down to the sub-molecular level) as “self”. The group of diseases can be divided in two categories, organ-specific and systemic diseases.

An “allergen” is defined as a substance, usually a macromolecule or a proteic composition which elicits the production of IgE antibodies in predisposed, particularly genetically disposed, individuals (atopics) patients. Similar definitions are presented in Liebers et al. (1996) Clin. Exp. Allergy 26, 494-516.

The term “therapeutically effective amount” refers to an amount of the peptide of the invention or derivative thereof, which produces the desired therapeutic or preventive effect in a patient. For example, in reference to a disease or disorder, it is the amount which reduces to some extent one or more symptoms of the disease or disorder, and more particularly returns to normal, either partially or completely, the physiological or biochemical parameters associated with or causative of the disease or disorder. Typically, the therapeutically effective amount is the amount of the peptide of the invention or derivative thereof, which will lead to an improvement or restoration of the normal physiological situation. For instance, when used to therapeutically treat a mammal affected by an immune disorder, it is a daily amount peptide/kg body weight of the said mammal. Alternatively, where the administration is through gene-therapy, the amount of naked DNA or viral vectors is adjusted to ensure the local production of the relevant dosage of the peptide of the invention, derivative or homologue thereof.

The term “natural” when referring to a peptide relates to the fact that the sequence is identical to a fragment of a naturally occurring protein (wild type or mutant). In contrast therewith the term “artificial” refers to a sequence which as such does not occur in nature. An artificial sequence is obtained from a natural sequence by limited modifications such as changing/deleting/inserting one or more amino acids within the naturally occurring sequence or by adding/removing amino acids N- or C-terminally of a naturally occurring sequence.

In this context, it is realised that peptide fragments are generated from antigens, typically in the context of epitope scanning. By coincidence such peptides may comprise in their sequence a T cell epitope (an MHC class II epitope or a CD1d binding epitope) and in their proximity a sequence with the modified redox motif as defined herein. Alternatively there can be an amino acid sequence of at most 11 amino acids, at most 7 amino acids, at most 4 amino acids, at most 2 amino acids between said epitope and said oxidoreductase motif, or even 0 amino acids (in other words the epitope and oxidoreductase motif sequence are immediately adjacent to each other).

In preferred embodiment, such naturally occurring peptides are disclaimed.

Amino acids are referred to herein with their full name, their three-letter abbreviation or their one letter abbreviation.

Motifs of amino acid sequences are written herein according to the format of Prosite. Motifs are used to describe a certain sequence variety at specific parts of a sequence. The symbol X is used for a position where any amino acid is accepted. Alternatives can be indicated by listing the acceptable amino acids for a given position, between square brackets (‘[ ]’). For example: [CST] stands for an amino acid selected from Cys, Ser or Thr. Amino acids which are excluded as alternatives can be indicated by listing them between curly brackets (‘{ }’). For example: {AM} stands for any amino acid except Ala and Met. The different elements in a motif are optionally separated from each other by a hyphen (-). The use of an integer between the symbol “X”, as in Xn indicates a numerical value or a numerical range of such amino acids. For example Xn, wherein n is an integer between 0 and 3 indicates that one of the following possibilities can occur within the motif: no X is present , a single X is present, 2 random amino acids X are present, or 3 random amino acids X are present. Xn wherein n is 2 hence corresponds to X-X or XX; while Xn, with n being 3 corresponds to X-X-X or XXX. Similarly, Bₘ, with B being any amino acid and m being an integer between 0 and 3 indicates either one of the options: no amino acid B is present, a single amino acid B is present, 2 random amino acids B are present, or 3 random amino acids B are present. The annotations “Z”, “Z¹” and/or “Z²” refer to a basic amino acid as defined herein elsewhere.

To distinguish between the amino acids, those outside the oxidoreductase motif can be called external amino acids, those within the redox motif are called internal amino acids.

A peptide, comprising a T cell epitope, e.g. an MHC class II T-cell epitope or an NKT-cell epitope (or CD1d binding peptide epitope) and a modified peptide motif sequence, having reducing activity is capable of generating a population of antigen-specific cytolytic CD4+ T-cells, respectively cytolytic NKT-cells towards antigen-presenting cells.

Accordingly, in its broadest sense, the invention relates to peptides which comprise at least one T-cell epitope (MHC class II T-cell epitope or an NKT-cell epitope) of an antigen (self or non-self) with a potential to trigger an immune reaction, and a modified thioreductase sequence motif with a reducing activity on peptide disulfide bonds. The T cell epitope and the modified redox motif sequence may be immediately adjacent to each other in the peptide or optionally separated by one or more amino acids (so called linker sequence). Optionally the peptide additionally comprises an endosome targeting sequence and/or additional “flanking” sequences.

The peptides of the invention comprise a T-cell epitope of an antigen (self or non self) with a potential to trigger an immune reaction, and a modified redox motif. The reducing activity of the motif sequence in the peptide can be assayed for its ability to reduce a sulfhydryl group such as in the insulin solubility assay wherein the solubility of insulin is altered upon reduction, or with a fluorescence-labelled substrate such as insulin. An example of such assay uses a fluorescent peptide and is described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

The modified redox motif may be positioned at the amino-terminus side of the T-cell epitope or at the carboxy-terminus of the T-cell epitope.

Peptide fragments with reducing activity are encountered in thioreductases which are small disulfide reducing enzymes including glutaredoxins, nucleoredoxins, thioredoxins and other thiol/disulfide oxidoreductases (Holmgren (2000) Antioxid. Redox Signal. 2, 811-820; Jacquot et al. (2002) Biochem. Pharm. 64, 1065-1069). They are multifunctional, ubiquitous and found in many prokaryotes and eukaryotes. They are known to exert reducing activity for disulfide bonds on proteins (such as enzymes) through redox active cysteines within conserved active domain consensus sequences well-known from e.g. Fomenko et al. ((2003) Biochemistry 42, 11214-11225; Fomenko et al. (2002) Prot. Science 11, 2285-2296), in which X stands for any amino acid. and WO2008/017517 comprising a cysteine at position 1 and/or 4. Thus the motif is either CXX[CST] or [CST]XXC. Such domains are also found in larger proteins such as protein disulfide isomerase (PDI) and phosphoinositide-specific phospholipase C. The present invention has redesigned said motifs in search for more potency and activity.

As explained in detail further on, the peptides of the present invention can be made by chemical synthesis, which allows the incorporation of non-natural amino acids. Accordingly, “C” in the above recited redox modified redox motifs represents either cysteine or another amino acid with a thiol group such as mercaptovaline, homocysteine or other natural or non-natural amino acids with a thiol function. In order to have reducing activity, the cysteines present in a modified redox motif should not occur as part of a cystine disulfide bridge. Nevertheless, a redox modified redox motif may comprise modified cysteines such as methylated cysteine, which is converted into cysteine with free thiol groups in vivo.

Peptides may further comprise modifications to increase stability or solubility, such as modification of the N-terminal NH₂ group or the C terminal COOH group (e.g. modification of the COOH into a CONH₂ group).

In the peptides of the present invention comprising a modified redox motif, the motif is located such that, when the epitope fits into the MHC groove, the motif remains outside of the MHC binding groove. The modified redox motif is placed either immediately adjacent to the epitope sequence within the peptide [in other words a linker sequence of zero amino acids between motif and epitope], or is separated from the T cell epitope by a linker comprising an amino acid sequence of 5 amino acids or less. More particularly, the linker comprises 1, 2, 3, 4, or 5 amino acids. Specific embodiments are peptides with a 0, 1 or 2 amino acid linker between epitope sequence and modified redox motif sequence. Apart from a peptide linker, other organic compounds can be used as linker to link the parts of the peptide to each other (e.g. the modified redox motif sequence to the T cell epitope sequence).

The peptides of the present invention can further comprise additional short amino acid sequences N or C-terminally of the sequence comprising the T cell epitope and the modified redox motif. Such an amino acid sequence is generally referred to herein as a ‘flanking sequence’. A flanking sequence can be positioned between the epitope and an endosomal targeting sequence and/or between the modified redox motif and an endosomal targeting sequence. In certain peptides, not comprising an endosomal targeting sequence, a short amino acid sequence may be present N and/or C terminally of the modified redox motif and/or epitope sequence in the peptide. More particularly a flanking sequence is a sequence of between 1 and 7 amino acids, most particularly a sequence of 2 amino acids.

The modified redox motif may be located N-terminal from the epitope.

In certain embodiments of the present invention, peptides are provided comprising one epitope sequence and a modified redox motif sequence. In further particular embodiments, the modified redox motif occurs several times (1, 2, 3, 4 or even more times) in the peptide, for example as repeats of the modified redox motif which can be spaced from each other by one or more amino acids or as repeats which are immediately adjacent to each other. Alternatively, one or more modified redox motifs are provided at both the N and the C terminus of the T cell epitope sequence.

Other variations envisaged for the peptides of the present invention include peptides which contain repeats of a T cell epitope sequence wherein each epitope sequence is preceded and/or followed by the modified redox motif (e.g. repeats of “modified redox motif-epitope” or repeats of “modified redox motif-epitope-modified redox motif”). Herein the modified redox motifs can all have the same sequence but this is not obligatory. It is noted that repetitive sequences of peptides which comprise an epitope which in itself comprises the modified redox motif will also result in a sequence comprising both the ‘epitope’ and a ‘modified redox motif’. In such peptides, the modified redox motif within one epitope sequence functions as a modified redox motif outside a second epitope sequence.

Typically the peptides of the present invention comprise only one T cell epitope. As described below a T cell epitope in a protein sequence can be identified by functional assays and/or one or more in silica prediction assays. The amino acids in a T cell epitope sequence are numbered according to their position in the binding groove of the MHC proteins. A T-cell epitope present within a peptide consist of between 7 and 30 amino acids, such as of between 8 and 25 amino acids, yet more particularly of between 8 and 16 amino acids, yet most particularly consists of 8, 9, 10, 11, 12, 13, 14, 15 or 16 amino acids.

In a more particular embodiment, the T cell epitope consists of a sequence of 9 amino acids. In a further particular embodiment, the T-cell epitope is an epitope, which is presented to T cells by MHC-class II molecules [MHC class II restricted T cell epitopes]. Typically T cell epitope sequence refers to the octapeptide or more specifically nonapeptide sequence which fits into the cleft of an MHC II protein.

In a more particular embodiment, the T cell epitope consists of a sequence of 7, 8, or 9 amino acids. In a further particular embodiment, the T-cell epitope is an epitope, which is presented by CD1d molecules [NKT cell epitopes]. Typically NKT cell epitope sequence refers to the 7 amino acid peptide sequence which binds to and is presented by the CD1d protein.

The T cell epitope of the peptides of the present invention can correspond either to a natural epitope sequence of a protein or can be a modified version thereof, provided the modified T cell epitope retains its ability to bind within the MHC cleft or to bind the CD1d receptor, similar to the natural T cell epitope sequence. The modified T cell epitope can have the same binding affinity for the MHC protein or the CD1d receptor as the natural epitope, but can also have a lowered affinity. In particular, the binding affinity of the modified peptide is no less than 10-fold less than the original peptide, more particularly no less than 5 times less. Peptides of the present invention have a stabilising effect on protein complexes. Accordingly, the stabilising effect of the peptide-MHC or CD1d complex compensates for the lowered affinity of the modified epitope for the MHC or CD1d molecule.

The sequence comprising the T cell epitope and the reducing compound within the peptide can be further linked to an amino acid sequence (or another organic compound) that facilitates uptake of the peptide into late endosomes for processing and presentation within MHC class II determinants. The late endosome targeting is mediated by signals present in the cytoplasmic tail of proteins and corresponds to well-identified peptide motifs. The late endosome targeting sequences allow for processing and efficient presentation of the antigen-derived T cell epitope by MHC-class II molecules. Such endosomal targeting sequences are contained, for example, within the gp75 protein (Vijayasaradhi et al. (1995) J. Cell. Biol. 130, 807-820), the human CD3 gamma protein, the HLA-BM 11 (Copier et al. (1996) J. Immunol. 157, 1017-1027), the cytoplasmic tail of the DEC205 receptor (Mahnke et al. (2000) J. Cell Biol. 151, 673-683). Other examples of peptides which function as sorting signals to the endosome are disclosed in the review of Bonifacio and Traub (2003) Annu. Rev. Biochem. 72, 395-447. Alternatively, the sequence can be that of a subdominant or minor T cell epitope from a protein, which facilitates uptake in late endosome without overcoming the T cell response towards the antigen. The late endosome targeting sequence can be located either at the amino-terminal or at the carboxy-terminal end of the antigen derived peptide for efficient uptake and processing and can also be coupled through a flanking sequence, such as a peptide sequence of up to 10 amino acids. When using a minor T cell epitope for targeting purpose, the latter is typically located at the amino-terminal end of the antigen derived peptide.

Alternatively, the present invention relates to the production of peptides containing hydrophobic residues that confer the capacity to bind to the CD1d molecule. Upon administration, such peptides are taken up by APC, directed to the late endosome where they are loaded onto CD1d and presented at the surface of the APC. Said hydrophobic peptides being characterized by a motif corresponding to the general sequence [FW]-xx-[ILM]-xx[FWTH] or [FWTH]-xx-[ILM]-xx-[FW,] in which positions P1 and P7 are occupied by hydrophobic residues such as phenylalanine (F) or tryptophan (W). P7 is however permissive in the sense that it accepts alternative hydrophobic residues to phenylalanine or tryptophan, such as threonine (T) or histidine (H). The P4 position is occupied by an aliphatic residue such as isoleucine (I), leucine (L) or methionine (M). The present invention relates to peptides made of hydrophobic residues which naturally constitute a CD1d binding motif. In some embodiment, amino acid residues of said motif are modified, usually by substitution with residues which increase the capacity to bind to 15 CD1d. In a specific embodiment, motifs are modified to fit more closely with the general motif [FW]-xx-[ILM]-xx-[FWTH]. More particularly, peptides are produced to contain a F or W at position 7.

Accordingly, the present invention envisages peptides of antigenic proteins and their use in eliciting specific immune reactions. These peptides can either correspond to fragments of proteins which comprise, within their sequence i.e. a reducing compound and a T cell epitope separated by at most 10, preferably 7 amino acids or less. Alternatively, and for most antigenic proteins, the peptides of the invention are generated by coupling a reducing compound, more particularly a reducing modified redox motif as described herein, N-terminally or C-terminally to a T cell epitope of the antigenic protein (either directly adjacent thereto or with a linker of at most 10, more particularly at most 7 amino acids). Moreover the T cell epitope sequence of the protein and/or the modified redox motif can be modified and/or one or more flanking sequences and/or a targeting sequence can be introduced (or modified), compared to the naturally occurring sequence. Thus, depending on whether or not the features of the present invention can be found within the sequence of the antigenic protein of interest, the peptides of the present invention can comprise a sequence which is ‘artificial’ or ‘naturally occurring’.

The peptides of the present invention can vary substantially in length. The length of the peptides can vary from 13 or 14 amino acids, i.e. consisting of an epitope of 8-9 amino acids, adjacent thereto the modified redox motif 5 amino acids with the histidine, up to 20, 25, 30, 40 or 50 amino acids. For example, a peptide may comprise an endosomal targeting sequence of 40 amino acids, a flanking sequence of about 2 amino acids, a motif as described herein of 5 amino acids, a linker of 4 amino acids and a T cell epitope peptide of 9 amino acids.

Accordingly, in particular embodiments, the complete peptide consists of between 13 amino acids up 20, 25, 30, 40, 50, 75 or 100 amino acids. More particularly, where the reducing compound is a modified redox motif as described herein, the length of the (artificial or natural) sequence comprising the epitope and modified redox motif optionally connected by a linker (referred to herein as ‘epitope-modified redox motif’ sequence), without the endosomal targeting sequence, is critical. The ‘epitope-modified redox motif’ more particularly has a length of 13, 14, 15, 16, 17, 18 or 19 amino acids. Such peptides of 13 or 14 to 19 amino acids can optionally be coupled to an endosomal targeting signal of which the size is less critical.

As detailed above, in particular embodiments, the peptides of the present invention comprise a reducing modified redox motif as described herein linked to a T cell epitope sequence.

In further particular embodiments, the peptides of the invention are peptides comprising T cell epitopes which do not comprise an amino acid sequence with redox properties within their natural sequence.

However, in alternative embodiments, the T cell epitope may comprise any sequence of amino acids ensuring the binding of the epitope to the MHC cleft or to the CD1d molecule. Where an epitope of interest of an antigenic protein comprises a modified redox motif such as described herein within its epitope sequence, the immunogenic peptides according to the present invention comprise the sequence of a modified redox motif as described herein and/or of another reducing sequence coupled N- or C- terminally to the epitope sequence such that (contrary to the modified redox motif present within the epitope, which is buried within the cleft) the attached modified redox motif can ensure the reducing activity.

Accordingly the T cell epitope and motif are immediately adjacent or separated from each other and do not overlap. To assess the concept of “immediately adjacent” or “separated”, the 8 or 9 amino acid sequence which fits in the MHC cleft or CD1d molecule is determined and the distance between this octapeptide or nonapeptide with the redox motif tetrapeptide or modified redox motif pentapeptide including histidine is determined.

Generally, the peptides of the present invention are not natural (thus no fragments of proteins as such) but artificial peptides which contain, in addition to a T cell epitope, a modified redox motif as described herein, whereby the modified redox motif is immediately separated from the T cell epitope by a linker consisting of up to seven, most particularly up to four or up to 2 amino acids.

It has been shown that upon administration (i.e. injection) to a mammal of a peptide comprising an oxidoreductase motif and an MHC class II T-cell epitope (or a composition comprising such a peptide), the peptide elicits the activation of T cells recognising the antigen derived T cell epitope and provides an additional signal to the T cell through reduction of surface receptor. This supra-optimal activation results in T cells acquiring cytolytic properties for the cell presenting the T cell epitope, as well as suppressive properties on bystander T cells.

Additionally, it has been shown that upon administration (i.e. injection) to a mammal of a peptide comprising an oxidoreductase motif and an NKT-cell epitope (or a composition comprising such a peptide), the peptide elicits the activation of T cells recognising the antigen derived T cell epitope and provides an additional signal to the T cell through binding to the CD1d surface receptor. This activation results in NKT cells acquiring cytolytic properties for the cell presenting the T cell epitope.

In this way, the peptides or composition comprising the peptides described in the present invention, which contain an antigen-derived T cell epitope and, outside the epitope, a modified redox motif can be used for direct immunisation of mammals, including human beings. The invention thus provides peptides of the invention or derivatives thereof, for use as a medicine. Accordingly, the present invention provides therapeutic methods which comprise administering one or more peptides according to the present invention to a patient in need thereof.

The present invention offers methods by which antigen-specific T cells endowed with cytolytic properties can be elicited by immunisation with small peptides. It has been found that peptides which contain (i) a sequence encoding a T cell epitope from an antigen and (ii) a consensus sequence with redox properties, and further optionally also comprising a sequence to facilitate the uptake of the peptide into late endosomes for efficient MHC-class II presentation or CD1d receptor binding, elicit cytolytic CD4+ T-cells or NKT cells respectively.

The immunogenic properties of the peptides of the present invention are of particular interest in the treatment and prevention of immune reactions.

Peptides described herein are used as medicament, more particularly used for the manufacture of a medicament for the prevention or treatment of an immune disorder in a mammal, more in particular in a human.

The present invention describes methods of treatment or prevention of an immune disorder of a mammal in need for such treatment or prevention, by using the peptides of the invention, homologues or derivatives thereof, the methods comprising the step of administering to said mammal suffering or at risk of an immune disorder a therapeutically effective amount of the peptides of the invention, homologues or derivatives thereof such as to reduce the symptoms of the immune disorder. The treatment of both humans and animals, such as, pets and farm animals is envisaged. In an embodiment the mammal to be treated is a human. The immune disorders referred to above are in a particular embodiment selected from allergic diseases and autoimmune diseases.

The peptides of the invention or the pharmaceutical composition comprising such as defined herein is preferably administered through sub-cutaneous or intramuscular administration. Preferably, the peptides or pharmaceutical compositions comprising such can be injected sub-cutaneously (SC) in the region of the lateral part of the upper arm, midway between the elbow and the shoulder. When two or more separate injections are needed, they can be administered concomitantly in both arms.

The peptide according to the invention or the pharmaceutical composition comprising such is administered in a therapeutically effective dose. Exemplary but non-limiting dosage regimens are between 50 and 1500 μg, preferably between 100 and 1200 μg. More specific dosage schemes can be between 50 and 250 μg, between 250 and 450 μg or between 850 and 1300 μg, depending on the condition of the patient and severity of disease. Dosage regimen can comprise the administration in a single dose or in 2, 3, 4, 5, or more doses, either simultaneously or consecutively. Exemplary non-limiting administration schemes are the following:
A low dose scheme comprising the SC administration of 50 μg of peptide in two separate injections of 25 μg each (100 μL each) followed by three consecutive injections of 25 μg of peptide as two separate injections of 12.5 μg each (50 μL each).
A medium dose scheme comprising the SC administration of 150 μg of peptide in two separate injections of 75 μg each (300 μL each) followed by three consecutive administrations of 75 μg of peptide as two separate injections of 37.5 μg each (150 μL each).
A high dose scheme comprising the SC administration of 450 μg of peptide in two separate injections of 225 μg each (900 μL each) followed by three consecutive administrations of 225 μg of peptide as two separate injections of 112.5 μg each (450 μL each).

An exemplary dose scheme of an immunogenic peptide comprising a known oxidoreductase motif and a T-cell epitope can be found on ClinicalTrials.gov under Identifier NCT03272269.

The present invention provides for immunogenic peptides comprising a new oxidoreductase motif and a T-cell epitope of an antigenic protein, optionally separated by a linker of between 0 and 7 amino acids.

Said new oxidoreductase motif is selected from the group comprising:

[CST]XₙC or CXₙ[CST],

wherein X is any amino acid, and

wherein n is an integer selected from the group comprising: 0, 1, 3, 4, 5 or 6.

In a preferred embodiment, said oxidoreductase motif is CC or CXC, wherein X can be any amino acid selected from the group consisting of: G, A, V, L, I, M, F, W, P, S, T, C, Y, N, Q, D, E, K, R, and H, or non-natural basic amino acids. Preferably, X in the CXC motif is any amino acid except for C, S, or T. In a specific embodiment, X in the CXC motif is a basic amino acid, such as H, K, or R, or a non-natural basic amino acid such as, but not limited to:
lysine variants like Fmoc-β-Lys(Boc)-OH (CAS Number 219967-68-7), Fmoc-Orn(Boc)-OH also called L-ornithine or ornithine (CAS Number 109425-55-0), Fmoc-β-Homolys(Boc)-OH (CAS Number 203854-47-1), Fmoc-Dap(Boc)-OH (CAS Number 162558-25-0) or Fmoc-Lys(Boc)OH(DiMe)-OH (CAS Number 441020-33-3);
tyrosine/phenylalanine variants like Fmoc-L-3Pal-OH (CAS Number 175453-07-3), Fmoc-β-HomoPhe(CN)-OH (CAS Number 270065-87-7), Fmoc-L-β-HomoAla(4-pyridyl)-OH (CAS Number 270065-69-5) or Fmoc-L-Phe(4-NHBoc)-OH (CAS Number 174132-31-1);
proline variants like Fmoc-Pro(4-NHBoc)-OH (CAS Number 221352-74-5) or Fmoc-Hyp(tBu)-OH (CAS Number 122996-47-8);
arginine variants like Fmoc-β-Homoarg(Pmc)-OH (CAS Number 700377-76-0).

Specific examples of the CXC motif are: CHC, CKC, CRC, CGC, CAC, CVC, CLC, CIC, CMC, CFC, CWC, CPC, CSC, CTC, CYC, CNC, CQC, CDC, and CEC.

In a preferred embodiment, said oxidoreductase motif is CX₃C, i.e. CXXXC, typically CX¹X²X³C, wherein X¹, X², and X³, each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², and X³ in said motif is any amino acid except for C, S, or T. In a specific embodiment, at least one of X¹, X², or X³ in said motif is a basic amino acid, such as H, K, or R, or a non-natural basic amino acid as defined herein.

Specific examples of the CXXXC motif are: CXPYC, CPXYC, and CPYXC, wherein X can be can be any amino acid, more preferably CXPYC, such as: CKPYC, CRPYC, CHPYC, CGPYC, CAPYC, CVPYC, CLPYC, CIPYC, CMPYC, CFPYC, CWPYC, CPPYC, CSPYC, CTPYC, CCPYC, CYPYC, CNPYC, CQPYC, CDPYC, CEPYC, and CKPYC; or

CPXYC, such as: CPKYC, CPRYC, CPHYC, CPGYC, CPAYC, CPVYC, CPLYC, CPIYC, CPMYC, CPFYC, CPWYC, CPPYC, CPSYC, CPTYC, CPCYC, CPYYC, CPNYC, CPQYC, CPDYC, CPEYC, and CPLYC; or CPYXC, such as: CPYKC, CPYRC, CPYHC, CPYGC, CPYAC, CPYVC, CPYLC, CPYIC, CPYMC, CPYFC, CPYWC, CPYPC, CPYSC, CPYTC, CPYCC, CPYYC, CPYNC, CPYQC, CPYDC, CPYEC, and CPYLC.

Further specific examples of the CXXXC motif are: CXHGC, CHXGC, and CHGXC, wherein X can be can be any amino acid, more preferably CXHGC, such as: CKHGC, CRHGC, CHHGC, CGHGC, CAHGC, CVHGC, CLHGC, CIHGC, CMHGC, CFHGC, CWHGC, CPHGC, CSHGC, CTHGC, CCHGC, CYHGC, CNHGC, CQHGC, CDHGC, CEHGC, and CKHGC; or

CGXHC, such as: CGKHC, CGRHC, CGHHC, CGGHC, CGAHC, CGVHC, CGLHC, CGIHC, CGMHC, CGFHC, CGWHC, CGPHC, CGSHC, CGTHC, CGCHC, CGYHC, CGNHC, CGQHC, CGDHC, CGEHC, and CGLHC; or

CHGXC, such as: CHGKC, CHGRC, CHGHC, CHGGC, CHGAC, CHGVC, CHGLC, CHGIC, CHGMC, CHGFC, CHGWC, CHGPC, CHGSC, CHGTC, CHGCC, CHGYC, CHGNC, CHGQC, CHGDC, CHGEC, and CHGLC.

Further specific examples of the CXXXC motif are: CXGPC, CGXPC, and CGPXC, wherein X can be can be any amino acid, more preferably CXGPC, such as: CKGPC, CRGPC, CHGPC, CGGPC, CAGPC, CVGPC, CLGPC, CIGPC, CMGPC, CFGPC, CWGPC, CPGPC, CSGPC, CTGPC, CCGPC, CYGPC, CNGPC, CQGPC, CDGPC, CEGPC, and CKGPC; or

CGXPC, such as: CGKPC, CGRPC, CGHPC, CGGPC, CGAPC, CGVPC, CGLPC, CGIPC, CGMPC, CGFPC, CGWPC, CGPPC, CGSPC, CGTPC, CGCPC, CGYPC, CGNPC, CGQPC, CGDPC, CGEPC, and CGLPC; or

CGPXC, such as: CGPKC, CGPRC, CGPHC, CGPGC, CGPAC, CGPVC, CGPLC, CGPIC, CGPMC, CGPFC, CGPWC, CGPPC, CGPSC, CGPTC, CGPCC, CGPYC, CGPNC, CGPQC, CGPDC, CGPEC, and CGPLC.

Further specific examples of the CXXXC motif are: CXGHC, CGXHC, and CGHXC, wherein X can be can be any amino acid, more preferably CXGHC, such as: CKGHC, CRGHC, CHGHC, CGGHC, CAGHC, CVGHC, CLGHC, CIGHC, CMGHC, CFGHC, CWGHC, CPGHC, CSGHC, CTGHC, CCGHC, CYGHC, CNGHC, CQGHC, CDGHC, CEGHC, and CKGHC; or

CGXFC, such as: CGKFC, CGRFC, CGHFC, CGGFC, CGAFC, CGVFC, CGLFC, CGIFC, CGMFC, CGFFC, CGWFC, CGPFC, CGSFC, CGTFC, CGCFC, CGYFC, CGNFC, CGQFC, CGDFC, CGEFC, and CGLFC; or CGHXC, such as: CGHKC, CGHRC, CGHHC, CGHGC, CGHAC, CGHVC, CGHLC, CGHIC, CGHMC, CGHFC, CGHWC, CGHPC, CGHSC, CGHTC, CGHCC, CGHYC, CGHNC, CGHQC, CGHDC, CGHEC, and CGHLC.

Further specific examples of the CXXXC motif are: CXGFC, CGXFC, and CGFXC, wherein X can be can be any amino acid, more preferably CXGFC, such as: CKGFC, CRGFC, CHGFC, CGGFC, CAGFC, CVGFC, CLGFC, CIGFC, CMGFC, CFGFC, CWGFC, CPGFC, CSGFC, CTGFC, CCGFC, CYGFC, CNGFC, CQGFC, CDGFC, CEGFC, and CKGFC; or

CGXFC, such as: CGKFC, CGRFC, CGHFC, CGGFC, CGAFC, CGVFC, CGLFC, CGIFC, CGMFC, CGFFC, CGWFC, CGPFC, CGSFC, CGTFC, CGCFC, CGYFC, CGNFC, CGQFC, CGDFC, CGEFC, and CGLFC; or CGFXC, such as: CGFKC, CGFRC, CGFHC, CGFGC, CGFAC, CGFVC, CGFLC, CGFIC, CGFMC, CGFFC, CGFWC, CGFPC, CGFSC, CGFTC, CGFCC, CGFYC, CGFNC, CGFQC, CGFDC, CGFEC, and CGFLC.

Further specific examples of the CXXXC motif are: CXRLC, CRXLC, and CRLXC, wherein X can be can be any amino acid, more preferably CXRLC, such as: CKRLC, CRRLC, CHRLC, CGRLC, CARLC, CVRLC, CLRLC, CIRLC, CMRLC, CFRLC, CWRLC, CPRLC, CSRLC, CTRLC, CCRLC, CYRLC, CNRLC, CQRLC, CDRLC, CERLC, and CKRLC; or

CRXLC, such as: CRKLC, CRRLC, CRHLC, CRGLC, CRALC, CRVLC, CRLLC, CRILC, CRMLC, CRFLC, CRWLC, CRPLC, CRSLC, CRTLC, CRCLC, CRYLC, CRNLC, CRQLC, CRDLC, CRELC, and CRLLC; or CRLXC, such as: CRLKC, CRLRC, CRLHC, CRLGC, CRLAC, CRLVC, CRLLC, CRLIC, CRLMC, CRLFC, CRLWC, CRLPC, CRLSC, CRLTC, CRLCC, CRLYC, CRLNC, CRLQC, CRLDC, CRLEC, and CRLLC.

Further specific examples of the CXXXC motif are: CXHPC, CHXPC, and CHPXC, wherein X can be can be any amino acid, more preferably CXHPC, such as: CKHPC, CRHPC, CHHPC, CGHPC, CAHPC, CVHPC, CLHPC, CIHPC, CMHPC, CFHPC, CWHPC, CPHPC, CSHPC, CTHPC, CCHPC, CYHPC, CNHPC, CQHPC, CDHPC, CEHPC, and CKHPC; or

CHXPC, such as: CHKPC, CHRPC, CHHPC, CHGPC, CHAPC, CHVPC, CHLPC, CHIPC, CHMPC, CHFPC, CHWPC, CHPPC, CHSPC, CHTPC, CHCPC, CHYPC, CHNPC, CHQPC, CHDPC, CHEPC, and CHLPC; or

CHPXC, such as: CHPKC, CHPRC, CHPHC, CHPGC, CHPAC, CHPVC, CHPLC, CHPIC, CHPMC, CHPFC, CHPWC, CHPPC, CHPSC, CHPTC, CHPCC, CHPYC, CHPNC, CHPQC, CHPDC, CHPEC, and CHPLC.

In a preferred embodiment, said oxidoreductase motif is CX₄C, i.e. CXXXXC, typically CX¹X²X³X⁴C, wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³ and X⁴ in said motif is any amino acid except for C, S, or T. In a specific embodiment, at least one of X¹, X², X³ or X⁴ in said motif is a basic amino acid, such as H, K, or R, or a non-natural basic amino acid as defined herein. Specific examples of the CXXXC motif are: CLAVLC, CTVQAC or CGAVHC and their variants such as: CX¹AVLC, CLX²VLC, CLAX³LC, or CLAVX⁴C; CX¹VQAC, CTX²QAC, CTVX³AC, or CTVQX⁴C; CX¹AVHC, CGX²VHC, CGAX³HC, or CGAVX⁴C; wherein X¹, X², X³ and X⁴ each individually can be any amino acid selected from the group consisting of: G, A, V, L, I, M, F, W, P, S, T, C, Y, N, Q, D, E, K, R, and H, or non-natural basic amino acids as defined herein.

In a preferred embodiment, said oxidoreductase motif is CX₅C, i.e. CXXXXXC, typically CX¹X²X³X⁴X⁵C, wherein X¹, X², X³, X⁴ and X⁵ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³, X⁴ and X⁵ in said motif is any amino acid except for C, S, or T. In a specific embodiment, at least one of X¹, X², X³ X⁴ or X⁵ in said motif is a basic amino acid, such as H, K, or R, or a non-natural basic amino acid as defined herein.

Specific examples of the CXXXXXC motif are: CPAFPLC or CDQGGEC and their variants such as: CX¹AFPLC, CPX²FPLC, CPAX³PLC, CPAFX⁴LC, or CPAFPX⁵C; CX¹QGGEC, CDX²GGEC, CDQX³GEC, CDQGX⁴EC, or CDQGGX⁵C, wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural basic amino acids as defined herein.

In a preferred embodiment, said oxidoreductase motif is CX₆C, i.e. CXXXXXXC, typically CX¹X²X³X⁴X⁵X⁶C, wherein X¹, X², X³, X⁴ X⁵ and X⁶ each individually can be any amino acid selected from the group consisting of: G, A, V, L, I, M, F, W, P, S, T, C, Y, N, Q, D, E, K, R, and H, or non-natural basic amino acids as defined herein. Preferably, X¹, X², X³, X⁴, X⁵ and X⁶ in said motif is any amino acid except for C, S, or T. In a specific embodiment, at least one of X¹, X², X³ X⁴, X⁵ or X⁶ in said motif is a basic amino acid, such as H, K, or R, or a non-natural basic amino acid as defined herein.

A specific example of the CXXXXXXC motif is: CDIADKYC or variants thereof such as: CX¹IADKYC, CDX²ADKYC, CDIX³DKYC, CDIAX⁴KYC, CDIADX⁵YC, or CDIADKX⁶C, wherein X¹, X², X³, X⁴, and X⁵ each individually can be any amino acid selected from the group consisting of: G, A, V, L, I, M, F, W, P, S, T, C, Y, N, Q, D, E, K, R, and H, or non-natural basic amino acids as defined herein,

In any one of these specific examples disclosed herein, the motif can additionally be flanked by a basic amino acid. Hence, such a basic amino acid (“Z”, “Z¹” and/or “Z²”) can be situated either before or after the [CST]XₙC or CXₙ[CST] motif and can be separated therefrom by no, one, two, or three random amino acids (B)—indicated as Bₘ, wherein m is an integer from 0 to 3. This will be resulting e.g. in any one of the following motifs: ZBₘ[CST]XₙC, [CST]XₙCBₘZ, ZBₘCXₙ[CST], or CXₙ[CST]BₘZ

wherein X or B is any amino acid,

wherein n is an integer selected from the group comprising: 0, 1, 3, 4, 5 or 6,

wherein m is an integer between 0 and 3,

wherein either one of, or each of “Z”, “Z¹” and/or “Z²” is a basic amino acid, preferably selected from the group comprising: K, H, R or a non-natural basic amino acid, more preferably wherein said basic amino acid is K or L-ornithine.

In any one of these specific examples, said oxidoreductase motif can be selected from the group comprising:

Z¹-Bₗ-8 CST]-Xₙ-C,

Z¹-Bₗ-C-Xₙ-[CST],

[CST]-Xₙ-C-Bₘ-Z²,

C-Xₙ-[CST]-Bₘ-Z²,

Z¹-Bₗ-[CST]-Xₙ-C-Bₘ-Z² or

Z¹-Bₗ-C-Xₙ-[CST]-Bₘ-Z²,

wherein each of X, Bₗ and Bₘ is any amino acid, preferably wherein at least one X in the motif is a basic amino acid preferably selected from the group comprising: K, H, R or a non-natural basic amino acid, further preferably wherein each of Bₗ and/or Bₘ is K, H, or R, more preferably H,

wherein n is an integer selected from the group comprising: 0, 1, 3, 4, 5 or 6,

wherein l and m are an integer selected from the group comprising 0 to 3,

wherein Z¹ and Z² are basic amino acids, preferably selected from the group comprising: K, H, R or a non-natural basic amino acid.

Particularly preferred examples of such oxidoreductase motifs are:

C[KHR]C, CX[KHR]XC, CXX[KHR]C, C[KHR]XXC, [KHR]CC, [KHR]CXC, [KHR]XXXC CC[KHR], CXC[KHR], CXXXC[KHR], [KHR]CC[KHR], [KHR]CXC[KHR], [KHR]CXXXC[KHR], [KHR]C[KHR]C, C[KHR]C[KHR], [KHR]CXX[KHR]C, [KHR]CX[KHR]XC, [KHR]C[KHR]XXC, CXX[KHR]C[KHR], CX[KHR]XC[KHR], C[KHR]XXC[KHR], and the like.

The peptides of the present invention can also be used in diagnostic in vitro methods for detecting class II restricted CD4 + T cells in a sample. In this method a sample is contacted with a complex of an MHC class II molecule and a peptide according to the present invention. The CD4+ T cells are detected by measuring the binding of the complex with cells in the sample, wherein the binding of the complex to a cell is indicative for the presence of CD4 + T cells in the sample. The complex can be a fusion protein of the peptide and an MHC class II molecule. Alternatively MHC molecules in the complex are tetramers. The complex can be provided as a soluble molecule or can be attached to a carrier.

The peptides of the present invention can also be used in diagnostic in vitro methods for detecting NKT cells in a sample. In this method a sample is contacted with a complex of a CD1d molecule and a peptide according to the present invention. The NKT cells are detected by measuring the binding of the complex with cells in the sample, wherein the binding of the complex to a cell is indicative for the presence of NKT cells in the sample. The complex can be a fusion protein of the peptide and a CD1d molecule.

Accordingly, in particular embodiments, the methods of treatment and prevention of the present invention comprise the administration of an immunogenic peptide as described herein, wherein the peptide comprise a T cell epitope of an antigenic protein which plays a role in the disease to be treated (for instance such as those described above). In further particular embodiments, the epitope used is a dominant epitope.

Peptides in accordance of the present invention will be prepared by synthesising a peptide wherein T cell epitope and modified redox motif will be separated by 0 to 5 amino acids. In certain embodiments the modified redox motif can be obtained by introducing 1, 2 or 3 mutations outside the epitope sequence, to preserve the sequence context as occurring in the protein. Typically amino-acids in P-2 and P-1, as well as in P+10 and P+11, with reference to the nonapeptide which are part of the natural sequence are preserved in the peptide sequence. These flanking residues generally stabilize the binding to MHC class II or CD1d molecules. In other embodiments the sequence N terminal or C terminal of the epitope will be unrelated to the sequence of the antigenic protein containing the T cell epitope sequence.

Thus based upon the above methods for designing a peptide, a peptide is generated by chemical peptide synthesis, recombinant expression methods or in more exceptional cases, proteolytic or chemical fragmentation of proteins.

Peptides as produced in the above methods can be tested for the presence of a T cell epitope in in vitro and in vivo methods, and can be tested for their reducing activity in in vitro assays. As a final quality control, the peptides can be tested in in vitro assays to verify whether the peptides can generate CD4+ T or NKT cells which are cytolytic via an apoptotic pathway for antigen presenting cells presenting the antigen which contains the epitope sequence which is also present in the peptide with the modified redox motif.

The peptides of the present invention can be generated using recombinant DNA techniques, in bacteria, yeast, insect cells, plant cells or mammalian cells. In view of the limited length of the peptides, they can be prepared by chemical peptide synthesis, wherein peptides are prepared by coupling the different amino acids to each other. Chemical synthesis is particularly suitable for the inclusion of e.g. D-amino acids, amino acids with non-naturally occurring side chains or natural amino acids with modified side chains such as methylated cysteine.

Chemical peptide synthesis methods are well described and peptides can be ordered from companies such as Applied Biosystems and other companies.

Peptide synthesis can be performed as either solid phase peptide synthesis (SPPS) or contrary to solution phase peptide synthesis. The best known SPPS methods are t-Boc and Fmoc solid phase chemistry:

During peptide synthesis several protecting groups are used. For example hydroxyl and carboxyl functionalities are protected by t-butyl group, lysine and tryptophan are protected by t-Boc group, and asparagine, glutamine, cysteine and histidine are protected by trityl group, and arginine is protected by the pbf group. If appropriate, such protecting groups can be left on the peptide after synthesis. Peptides can be linked to each other to form longer peptides using a ligation strategy (chemoselective coupling of two unprotected peptide fragments) as originally described by Kent (Schnelzer & Kent (1992) Int. J. Pept. Protein Res. 40, 180-193) and reviewed for example in Tam et al. (2001) Biopolymers 60, 194-205 provides the tremendous potential to achieve protein synthesis which is beyond the scope of SPPS. Many proteins with the size of 100-300 residues have been synthesised successfully by this method. Synthetic peptides have continued to play an ever increasing crucial role in the research fields of biochemistry, pharmacology, neurobiology, enzymology and molecular biology because of the enormous advances in the SPPS. Alternatively, the peptides can be synthesised by using nucleic acid molecules which encode the peptides of this invention in an appropriate expression vector which include the encoding nucleotide sequences. Such DNA molecules may be readily prepared using an automated DNA synthesiser and the well-known codon-amino acid relationship of the genetic code. Such a DNA molecule also may be obtained as genomic DNA or as cDNA using oligonucleotide probes and conventional hybridisation methodologies. Such DNA molecules may be incorporated into expression vectors, including plasmids, which are adapted for the expression of the DNA and production of the polypeptide in a suitable host such as bacterium, e.g. Escherichia coli, yeast cell, animal cell or plant cell.

The physical and chemical properties of a peptide of interest (e.g. solubility, stability) are examined to determine whether the peptide is/would be suitable for use in therapeutic compositions. Typically this is optimised by adjusting the sequence of the peptide. Optionally, the peptide can be modified after synthesis (chemical modifications e.g. adding/deleting functional groups) using techniques known in the art.

The mechanism of action of immunogenic peptides comprising a standard oxidoreductase motif and an MHC class II T-cell epitope is substantiated with experimental data disclosed in the above cited PCT application WO2008/017517 and publications of the present inventors. The mechanism of action of immunogenic peptides comprising a standard oxidoreductase motif and a CD1d binding NKT-cell epitope is substantiated with experimental data disclosed in the above cited PCT application WO2012/069568 and publications of the present inventors.

The present invention provides methods for generating antigen-specific cytolytic CD4+ T-cells (when using an immunogenic peptide as disclosed herein comprising an MHC class II epitope), or antigen-specific cytolytic NKT-cells (when using an immunogenic peptide as disclosed herein comprising an NKT cell epitope binding the CD1d molecule) either in vivo or in vitro.

The present invention describes in vivo methods for the production of the antigen-specific CD4+ T cells or NKT cells. A particular embodiment relates to the method for producing or isolating the CD4+ T cells or NKT cells by immunising animals (including humans) with the peptides of the invention as described herein and then isolating the CD4+ T cells or NKT cells from the immunised animals. The present invention describes in vitro methods for the production of antigen specific cytolytic CD4+ T cells or NKT cells towards APC. The present invention provides methods for generating antigen specific cytolytic CD4 + T cells and NKT cells towards APC.

In one embodiment, methods are provided which comprise the isolation of peripheral blood cells, the stimulation of the cell population in vitro by an immunogenic peptide according to the invention and the expansion of the stimulated cell population, more particularly in the presence of IL-2. The methods according to the invention have the advantage a high number of CD4+ T cells is produced and that the CD4+ T cells can be generated which are specific for the antigenic protein (by using a peptide comprising an antigen-specific epitope).

In an alternative embodiment, the CD4+ T cells can be generated in vivo, i.e. by the injection of the immunogenic peptides described herein to a subject, and collection of the cytolytic CD4+ T cells generated in vivo.

The antigen-specific cytolytic CD4+ T cells towards APC, obtainable by the methods of the present invention are of particular interest for the administration to mammals for immunotherapy, in the prevention of allergic reactions and the treatment of auto-immune diseases. Both the use of allogenic and autogeneic cells are envisaged.

Cytolytic CD4+ T cells populations are obtained as described herein below.

In one embodiment, the invention provides ways to expand specific NKT cells, with as a consequence increased activity comprising, but not limited to:

(i) increased cytokine production

(ii) increased contact- and soluble factor-dependent elimination of antigen-presenting cells. The result is therefore a more efficient response towards intracellular pathogens, autoantigens, allofactors, allergens, tumor cells and more efficient suppression of immune responses against graft and viral proteins used in gene therapy/gene vaccination.

The present invention also relates to the identification of NKT cells with required properties in body fluids or organs. The method comprises identification of NKT cells by virtue of their surface phenotype, including expression of NK1.1, CD4, NKG2D and CD244. Cells are then contacted with NKT cell epitopes defined as peptides able to be presented by the CD1d molecule. Cells are then expanded in vitro in the presence of IL-2 or IL-15 or IL-7.

Antigen-specific cytolytic CD4+ T cells or NKT cells as described herein can be used as a medicament, more particularly for use in adoptive cell therapy, more particularly in the treatment of acute allergic reactions and relapses of autoimmune diseases such as multiple sclerosis. Isolated cytolytic CD4+ T cells or NKT cells or cell populations, more particularly antigen-specific cytolytic CD4+ T cell or NKT cell populations generated as described are used for the manufacture of a medicament for the prevention or treatment of immune disorders. Methods of treatment by using the isolated or generated cytolytic CD4+ T cells or NKT cells are disclosed.

As explained in WO2008/017517 cytolytic CD4+ T cells towards APC can be distinguished from natural Treg cells based on expression characteristics of the cells. More particularly, a cytolytic CD4 + T cell population demonstrates one or more of the following characteristics compared to a natural Treg cell population:

an increased expression of surface markers including CD103, CTLA-4, Fasl and ICOS upon activation, intermediate expression of CD25, expression of CD4, ICOS, CTLA-4, GITR and low or no expression of CD127 (IL7-R), no expression of CD27, expression of transcription factor T-bet and egr-2 (Krox-20) but not of the transcription repressor Foxp3, a high production of IFN-gamma and no or only trace amounts of IL-10, IL-4, IL-5, IL-13 or TGF-beta.

Further the cytolytic T cells express CD45RO and/or CD45RA, do not express CCR7, CD27 and present high levels of granzyme B and other granzymes as well as Fas ligand.

As explained in WO2008/017517 cytolytic NKT cells against towards APC can be distinguished from non-cytolytic NKT cells based on expression characteristics of the cells. More particularly, a cytolytic CD4 + NKT cell population demonstrates one or more of the following characteristics compared to a non-cytolytic NKT cell population: expression of NK1.I, CD4, NKG2D and CD244.

The peptides of the invention will, upon administration to a living animal, typically a human being, elicit specific T cells exerting a suppressive activity on bystander T cells.

In specific embodiments the cytolytic cell populations of the present invention are characterised by the expression of FasL and/or Interferon gamma. In specific embodiments the cytolytic cell populations of the present invention are further characterised by the expression of GranzymeB.

This mechanism also implies and the experimental results show that the peptides of the invention, although comprising a specific T-cell epitope of a certain antigen, can be used for the prevention or treatment of disorders elicited by an immune reaction against other T-cell epitopes of the same antigen or in certain circumstances even for the treatment of disorders elicited by an immune reaction against other T-cell epitopes of other different antigens if they would be presented through the same mechanism by MHC class II molecules or CD1d moelcules in the vicinity of T cells activated by peptides of the invention.

Isolated cell populations of the cell type having the characteristics described above, which, in addition are antigen-specific, i.e. capable of suppressing an antigen-specific immune response are disclosed.

The present invention provides pharmaceutical compositions comprising one or more peptides according to the present invention, further comprising a pharmaceutically acceptable carrier. As detailed above, the present invention also relates to the compositions for use as a medicine or to methods of treating a mammal of an immune disorder by using the composition and to the use of the compositions for the manufacture of a medicament for the prevention or treatment of immune disorders. The pharmaceutical composition could for example be a vaccine suitable for treating or preventing immune disorders, especially airborne and foodborne allergy, as well as diseases of allergic origin. As an example described further herein of a pharmaceutical composition, a peptide according to the invention is adsorbed on an adjuvant suitable for administration to mammals, such as aluminium hydroxide (alum). Typically, 50 μg of the peptide adsorbed on alum are injected by the subcutaneous route on 3 occasions at an interval of 2 weeks. It should be obvious for those skilled in the art that other routes of administration are possible, including oral, intranasal or intramuscular. Also, the number of injections and the amount injected can vary depending on the conditions to be treated. Further, other adjuvants than alum can be used, provided they facilitate peptide presentation in MHC-class II presentation and T cell activation. Thus, while it is possible for the active ingredients to be administered alone, they typically are presented as pharmaceutical formulations. The formulations, both for veterinary and for human use, of the present invention comprise at least one active ingredient, as above described, together with one or more pharmaceutically acceptable carriers. The present invention relates to pharmaceutical compositions, comprising, as an active ingredient, one or more peptides according to the invention, in admixture with a pharmaceutically acceptable carrier. The pharmaceutical composition of the present invention should comprise a therapeutically effective amount of the active ingredient, such as indicated hereinafter in respect to the method of treatment or prevention. Optionally, the composition further comprises other therapeutic ingredients. Suitable other therapeutic ingredients, as well as their usual dosage depending on the class to which they belong, are well known to those skilled in the art and can be selected from other known drugs used to treat immune disorders.

The term “pharmaceutically acceptable carrier” as used herein means any material or substance with which the active ingredient is formulated in order to facilitate its application or dissemination to the locus to be treated, for instance by dissolving, dispersing or diffusing the composition, and/or to facilitate its storage, transport or handling without impairing its effectiveness. They include any and all solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the immunogenic peptide in the composition. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders. Suitable pharmaceutical carriers for use in the pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals. The pharmaceutical compositions of the present invention may be prepared in any known manner, for instance by homogeneously mixing, coating and/or grinding the active ingredients, in a one- step or multi-steps procedure, with the selected carrier material and, where appropriate, the other additives such as surface-active agents. They may also be prepared by micronisation, for instance in view to obtain them in the form of microspheres usually having a diameter of about 1 to 10 μm, namely for the manufacture of microcapsules for controlled or sustained release of the active ingredients. Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water- soluble soaps and water-soluble synthetic surface-active agents. Suitable soaps are alkaline or alkaline-earth metal salts, unsubstituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives typically contain 8 to 22 carbon atoms. Examples of alkylarylsulphonates are the sodium, calcium or alcanolamine salts of dodecyl benzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl- ethanolamine, phosphatidylserine, phosphatidylglycerine, lysolecithin, cardiolipin, dioctanylphosphatidylcholine, dipalmitoylphoshatidylcholine and their mixtures. Suitable non-ionic surfactants include polyethoxylated and poly propoxylated derivatives of alkyl phenols, fatty alcohols, fatty acids, aliphatic amines or amides containing at least 12 carbon atoms in the molecule, alkylarene sulphonates and dialkylsulphosuccinates, such as polyglycol ether derivatives of aliphatic and cycloaliphatic alcohols, saturated and unsaturated fatty acids and alkylphenols, the derivatives typically containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol—polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants. Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbucw’, 2 d ed. (Hanser Verlag, Vienna, 1981) and “Encyclopaedia of Surfactants, (Chemical Publishing Co., New York, 1981). Peptides, homologues or derivatives thereof according to the invention (and their physiologically acceptable salts or pharmaceutical compositions all included in the term “active ingredients”) may be administered by any route appropriate to the condition to be treated and appropriate for the compounds, here the proteins and fragments to be administered. Possible routes include regional, systemic, oral (solid form or inhalation), rectal, nasal, topical (including ocular, buccal and sublingual), vaginal and parenteral (including subcutaneous, intramuscular, intravenous, intradermal, intra-arterial, intrathecal and epidural). The preferred route of administration may vary with for example the condition of the recipient or with the diseases to be treated. As described herein, the carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intraarterial, intrathecal and epidural) administration.

Formulations suitable for parenteral administration include aqueous and non-aqueous sterile injection solutions which may contain anti- oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Typical unit dosage formulations are those containing a daily dose or unit daily sub-dose, as herein above recited, or an appropriate fraction thereof, of an active ingredient. It should be understood that in addition to the ingredients particularly mentioned above the formulations of this invention may include other agents conventional in the art having regard to the type of formulation in question, for example those suitable for oral administration may include flavouring agents. Peptides, homologues or derivatives thereof according to the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polyniethyl methacrylate and the other above- described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injection include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof. In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Cytolytic CD4+ T cells as obtained in the present invention, induce APC apoptosis after MHC-class II dependent cognate activation, affecting both dendritic and B cells, as demonstrated in vitro and in vivo, and (2) suppress bystander T cells by a contact-dependent mechanism in the absence of IL-10 and/or TGF-beta. Cytolytic CD4+ T cells can be distinguished from both natural and adaptive Tregs, as discussed in detail in WO2008/017517.

The immunogenic peptides of the invention containing hydrophobic residues that confer the capacity to bind to the CD1d molecule. Upon administration, are taken up by APC, directed to the late endosome where they are loaded onto CD1d and presented at the surface of the APC. Once presented by CD1d molecule, the thioreductase motif in the peptides enhances the capacity to activate NKT cells, becoming cytolytic NKT cells. Said immunogenic peptides activate the production of cytokine, such as IFN-gamma, which will activate other effector cells including CD4+ T cells andnCD8+ T cells. Both CD4+ and CD8+ T cells can participate in the elimination of the cell presenting the antigen as discussed in detail in WO2012/069568.

The present invention will now be illustrated by means of the following examples which are provided without any limiting intention. Furthermore, all references described herein are explicitly included herein by reference.

### EXAMPLES

### Example 1: Methodology to Assess the Reducing Activity of Immunogenic Peptides

The oxidoreductase activity of the immunogenic peptides is determined using a fluorescent assay described in Tomazzolli et al. (2006) Anal. Biochem. 350, 105-112. Two peptides with a FITC label become self-quenching when they covalently attached to each other via a disulfide bridge. Upon reduction by a peptide in accordance with the present invention, the reduced individual peptides become fluorescent again.

### Example 2: Influence of the Oxidoreductase Motif (KCC) on the Redox Activity of Immunogenic Peptides

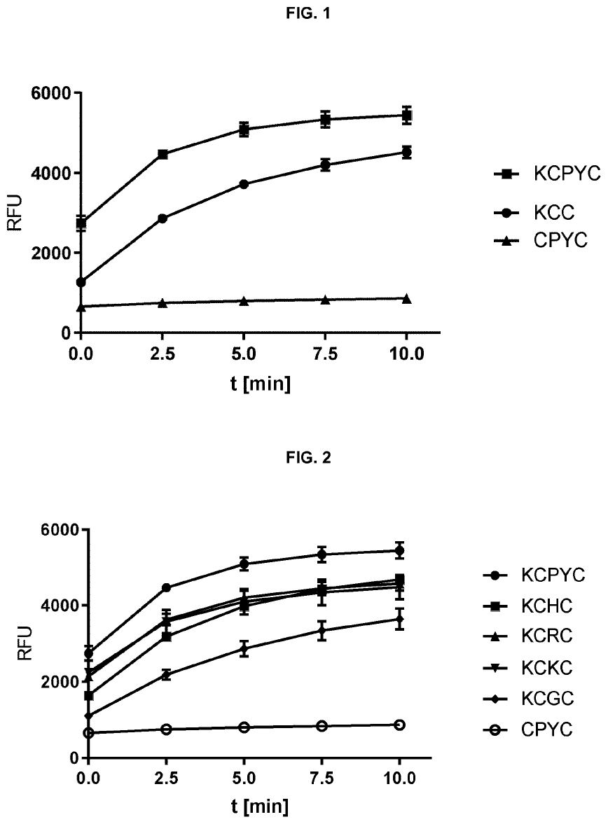

The following table 1 represents the peptide sequences which were used to test the redox activity of immunogenic peptides comprising a T cell epitope of the Tetanus Toxin protein and an oxidoreductase motif comprising two cysteines without any amino acid between the cysteines and a basic amino acid at the N-terminus of the motif (KCC motif). All the tests with these peptides were performed in duplicates, and each test was conducted two times. FIG. 1 represents the initial velocities of one repeat. The activity is expressed as the mean of duplicates. The results are expressed in Relative Fluorescent Units (RFU). Peptides with an oxidoreductase motif comprising the KCC sequence exhibited a better oxidoreductase activity than the control peptide with the CPYC motif, but lower than the control peptides with the KCPYC sequence (see table 1 and FIG. 1).

TABLE 1
influence of the redox motif (KCC) on the oxidoreductase activity
of an immunogenic peptide comprising a tetanus toxin T cell epitope.

| SEQ ID # | N-term | Motif | Linker | T-Epitope | C-term | Purpose |
|---|---|---|---|---|---|---|
| 1 | K | CC | V | QYIKANSKFIGIT | EL | Test peptide |
| 2 | K | CPYC | V | QYIKANSKFIGIT | EL | Control |
| 3 | | CPYC | V | QYIKANSKFIGIT | EL | Control |

### Example 3: Influence of the Oxidoreductase Motif (KCxC) on the Redox Activity of Immunogenic Peptides

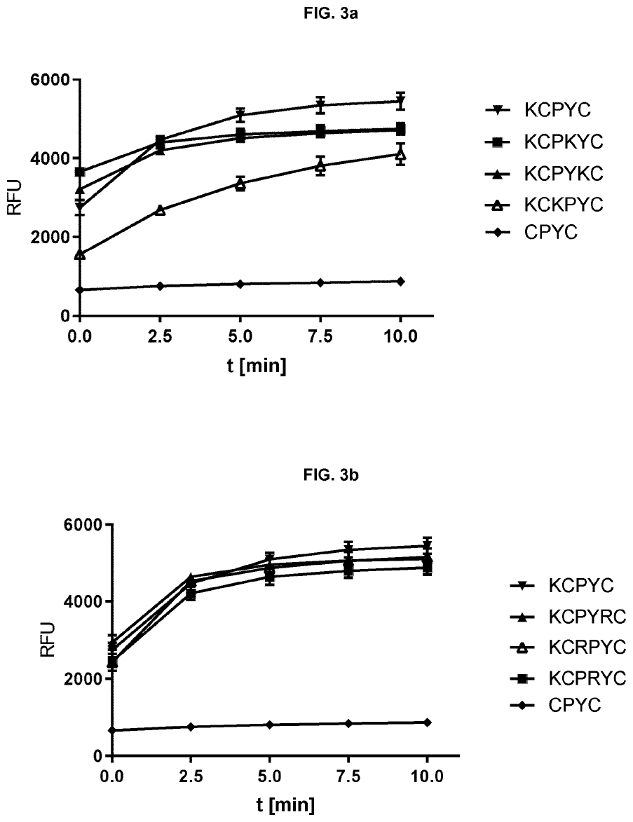

The following table 2 represents the peptide sequences which were used to test the redox activity of immunogenic peptides comprising a T cell epitope of the Tetanus Toxin protein and an oxidoreductase motif comprising two cysteines with one amino acid between the cysteines, and a basic amino acid at the N-terminus of the motif (KCxC motif). Tested peptides comprise a KCxC motif wherein x is a basic amino acid (H, K or R) or is G. All the tests with these peptides were performed in duplicates, and each test was conducted two times. FIG. 2 represents the initial velocities of one repeat. The activity is expressed as the mean of duplicates. The results are expressed in Relative Fluorescent Units (RFU). Peptides with an oxidoreductase motif comprising the KCxC sequence exhibited a better oxidoreductase activity than the control peptide with the CPYC motif, but lower than the control peptide with the KCPYC sequence (see table 2 and FIG. 2).

TABLE 2
Influence of the redox motif (KCxC) on the oxidoreductase activity
of an immunogenic peptide comprising a tetanus toxin T cell epitope.

| SEQ ID# | N-term | Motif | Linker | T-Epitope | C-term | Purpose |
|---|---|---|---|---|---|---|
| 4 | K | CHC | V | QYIKANSKFIGIT | EL | Test peptide |
| 5 | K | CKC | V | QYIKANSKFIGIT | EL | Test peptide |
| 6 | K | CRC | V | QYIKANSKFIGIT | EL | Test peptide |
| 7 | K | CGC | V | QYIKANSKFIGIT | EL | Test peptide |
| 8 | K | CPYC | V | QYIKANSKFIGIT | EL | Control |
| 3 | | CPYC | V | QYIKANSKFIGIT | EL | Control |

### Example 4: Influence of the Oxidoreductase Motif (KCxxxC) on the Redox Activity of Immunogenic Peptides

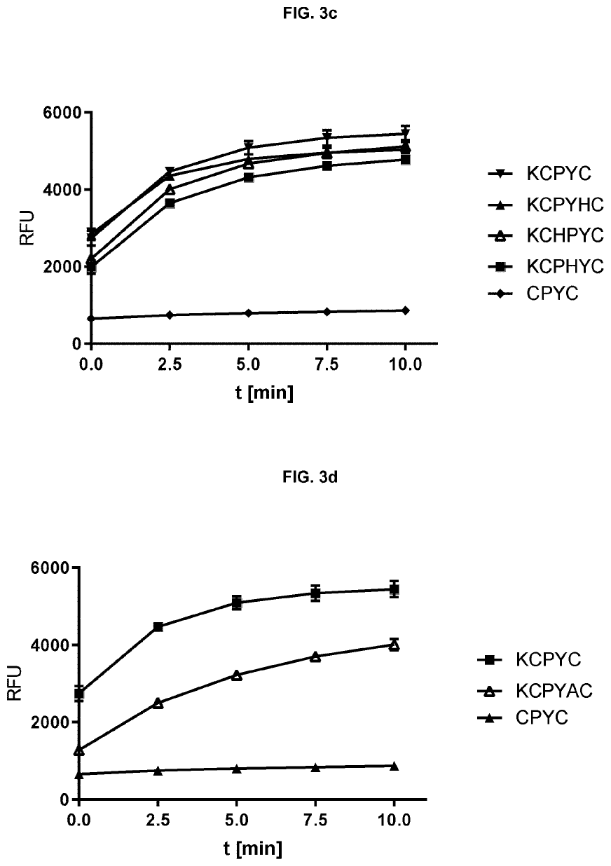

The following table 3a represents the peptide sequences which were used to test the redox activity of immunogenic peptides comprising a T cell epitope of the Tetanus Toxin protein and an oxidoreductase motif comprising two cysteines with three amino acid between the cysteines and a basic amino acid at the N-terminus of the motif (KCxxxC motif), wherein one x is the basic amino acid K. All the tests with these peptides were performed in duplicates, and each test was conducted two times. FIG. 3a represents the initial velocities of one repeat. The activity is expressed as the mean of duplicates. The results are expressed in Relative Fluorescent Units (RFU). Peptides with an oxidoreductase motif comprising the KCxxxC sequence wherein one x is the basic amino acid K exhibited a better oxidoreductase activity than the control peptide with the CPYC motif, and pretty much the same oxidoreductase activity than the KCPYC sequence, except for the KCKPYC sequence which seems less efficient (see table 3a and FIG. 3a).

TABLE 3a
influence of the redox motif (CxxxC) wherein one x is K on the
oxidoreductase activity of an immunogenic peptide comprising
a tetanus toxin T cell epitope.

| SEQ ID # | N-term | Motif | Linker | T-Epitope | C-term | Purpose |
|---|---|---|---|---|---|---|
| 9 | K | CKPYC | V | QYIKANSKFIGIT | EL | Test peptide |
| 10 | K | CPKYC | V | QYIKANSKFIGIT | EL | Test peptide |
| 11 | K | CPYKC | V | QYIKANSKFIGIT | EL | Test peptide |
| 12 | K | CPYC | V | QYIKANSKFIGIT | EL | Control |
| 3 | | CPYC | V | QYIKANSKFIGIT | EL | Control |

The following table 3b represents the peptide sequences which were used to test the redox activity of immunogenic peptides comprising a T cell epitope of the Tetanus Toxin protein and an oxidoreductase motif comprising two cysteines with three amino acid between the cysteines and a basic amino acid at the N-terminus of the motif (KCxxxC motif), wherein one x is the basic amino acid R. All the tests with these peptides were performed in duplicates, and each test was conducted two times. FIG. 3b represents the initial velocities of one repeat. The activity is expressed as the mean of duplicates. The results are expressed in Relative Fluorescent Units (RFU). Peptides with an oxidoreductase motif comprising the KCxxxC sequence wherein one x is the basic amino acid R exhibited a better oxidoreductase activity than the control peptide with the CPYC motif, and exhibited the same oxidoreductase activity than the control peptides with the KCPYC sequence (see table 3b and FIG. 3b).

TABLE 3b
Influence of the redox motif (KCxxxC) wherein one x is R on the
oxidoreductase activity of an Imotope containing tetanus toxin epitope

| SEQ ID# | N-term | Motif | Linker | T-Epitope | C-term | Purpose |
|---|---|---|---|---|---|---|
| 13 | K | CRPYC | V | QYIKANSKFIGIT | EL | Test peptide |
| 14 | K | CPRYC | V | QYIKANSKFIGIT | EL | Test peptide |
| 15 | K | CPYRC | V | QYIKANSKFIGIT | EL | Test peptide |
| 16 | K | CPYC | V | QYIKANSKFIGIT | EL | Control |
| 3 | | CPYC | V | QYIKANSKFIGIT | EL | Control |

The following table 3c represents the peptide sequences which were used to test the redox activity of immunogenic peptides comprising a T cell epitope of the Tetanus Toxin protein and an oxidoreductase motif comprising two cysteines with three amino acid between the cysteines and a basic amino acid at the N-terminus of the motif (KCxxxC motif), wherein one x is the basic amino acid H. All the tests with these peptides were performed in duplicates, and each test was conducted two times. FIG. 3c represents the initial velocities of one repeat. The activity is expressed as the mean of duplicates. The results are expressed in Relative Fluorescent Units (RFU). Peptides with an oxidoreductase motif comprising the KCxxxC sequence wherein one x is the basic amino acid H exhibited a better oxidoreductase activity than the control peptide with the CPYC motif, and exhibited pretty much the same oxidoreductase activity than the control peptides with the KCPYC sequence (see table 3c and FIG. 3c).

TABLE 3c
influence of the redox motif (KCxxxC) wherein one x is H on the oxidoreductase
activity of an immunogenic peptide comprising a tetanus toxin T cell epitope.

| SEQ ID# | N-term | Motif | Linker | T-Epitope | C-term | Purpose |
|---|---|---|---|---|---|---|
| 17 | K | CHPYC | V | QYIKANSKFIGIT | EL | Test peptide |
| 18 | K | CPHYC | V | QYIKANSKFIGIT | EL | Test peptide |
| 19 | K | CPYHC | V | QYIKANSKFIGIT | EL | Test peptide |
| 20 | K | CPYC | V | QYIKANSKFIGIT | EL | Control |
| 3 | | CPYC | V | QYIKANSKFIGIT | EL | Control |

The following table 3d represents the peptide sequences which were used to test the redox activity of immunogenic peptides comprising a T cell epitope of the Tetanus Toxin protein and an oxidoreductase motif comprising two cysteines with three amino acid between the cysteines and a basic amino acid at the N-terminus of the motif (KCxxxC motif), wherein the third x is the amino acid A. All the tests with these peptides were performed in duplicates, and each test was conducted two times. FIG. 3d represents the initial velocities of one repeat. The activity is expressed as the mean of duplicates. The results are expressed in Relative Fluorescent Units (RFU). Peptides with an oxidoreductase motif comprising the KCxxxC sequence wherein the third x is the amino acid A exhibited a better oxidoreductase activity than the control peptide with the CPYC motif, but lower than the control peptides with the KCPYC sequence (see table 3d and FIG. 3d).

TABLE 3d
Influence of the redox motif (KCxxxC) wherein the 3^{rd} X is an A on the
oxidoreductase activity of an Imotope containing tetanus toxin epitope.

| SEQ ID# | N-term | Motif | Linker | T-Epitope | C-term | Purpose |
|---|---|---|---|---|---|---|
| 21 | K | CPYAC | V | QYIKANSKFIGIT | EL | Test peptide |
| 22 | K | CPYC | V | QYIKANSKFIGIT | EL | Control |
| 3 | | CPYC | V | QYIKANSKFIGIT | EL | Control |

1-20. (canceled)

21. An immunogenic peptide, said immunogenic peptide comprising:
a) an oxidoreductase motif,
b) a T-cell epitope of an antigenic protein,
c) a linker between a) and b) of between 0 and 7 amino acids,
wherein said oxidoreductase motif comprises [CST]XₙC or CXₙ[CST], wherein n is 0, 1 or 3, wherein each of X is independently any amino acid with the proviso that at least one X in said oxidoreductase motif is a basic amino acid selected from the group consisting of K, H, R and a non-natural basic amino acid, and
wherein said oxidoreductase motif does not naturally occur within a region of 11 amino acids N-terminally or C-terminally of the T-cell epitope in said antigenic protein.

22. The immunogenic peptide according to claim 21, wherein said oxidoreductase motif comprises
(i) Z¹-Bₗ-[CST]-Xₙ-C or Z¹-Bₗ-C-Xₙ-[CST], wherein each of X and Bₗ is independently any amino acid, wherein Z¹ is a basic amino acid selected from the group consisting of K, H, R and a non-natural basic amino acid, wherein n is 0, 1 or 2, and wherein 1 is 0, 1, 2 or 3;
(ii) [CST]-Xₙ-C-Bₘ-Z² or C-Xₙ-[CST]-Bₘ-Z², wherein each of X and Bₘ is independently any amino acid, wherein Z² is a basic amino acid selected from the group consisting of K, H, R or a non-natural basic amino acid, wherein n is 0, 1 or 3, wherein m is 0, 1, 2 or 3; or
(iii) Z¹-Bₗ-[CST]-Xₙ-C-Bₘ-Z² or Z¹-Bₗ-C-Xₙ-[CST]-Bₘ-Z², wherein each of X, Bₗ and Bₘ is independently any amino acid, wherein Z¹ and Z² are independently basic amino acids selected from the group consisting of K, H, R or a non-natural basic amino acid, wherein n is 0, 1 or 3, wherein 1 and m are independently 0, 1, 2 or 3.

23. The immunogenic peptide according to claim 21, wherein said immunogenic peptide comprises a single oxidoreductase motif, wherein said oxidoreductase motif comprises Z¹-Bₗ-[CST]-Xₙ-C or Z¹-Bₗ-C-Xₙ-[CST],
wherein each of X and Bₗ is independently any amino acid,
wherein Z¹ is K or R,
wherein n is 1 or 3, and
wherein 1 is 0, 1, 2, or 3.

24. The immunogenic peptide according to claim 21, wherein said oxidoreductase motif is selected from the group consisting of C[KHR]C, C[KHR]XXC, CX[KHR]XC, CXX[KHR]C, [KHR]C[KHR]C, [KHR]C[KHR]XXC, [KHR]CX[KHR]XC, and [KHR]CXX[KHR]C.

25. The immunogenic peptide according to claim 21, wherein said T cell epitope of the antigenic protein is an NKT cell epitope or an MHC class II T cell epitope.

26. The immunogenic peptide according to claim 21, wherein said epitope has a length of between 7 and 25 amino acids and/or wherein said immunogenic peptide has a length of between 9 and 50 amino acids.

27. The immunogenic peptide according to claim 21, wherein said antigenic protein is an auto-antigen, a soluble allofactor, an alloantigen shed by a graft, an antigen of an intracellular pathogen, an antigen of a viral vector used for gene therapy or gene vaccination, a tumor-associated antigen or an allergen.

28. The immunogenic peptide according to claim 21, wherein the linker is of between 0 and 4 amino acids.

29. The immunogenic peptide according to claim 21, wherein the T-cell epitope does not naturally comprise said oxidoreductase motif.

30. A method of treating an autoimmune disease, an infection with an intracellular pathogen, a tumor, an allograft rejection, or suppressing an immune response to a soluble allofactor, allergen exposure or viral vector used for gene therapy or gene vaccination in a subject in need thereof comprising administering to the subject a therapeutically effective amount of the immunogenic peptide according to claim 21.

31. A method for preparing an immunogenic peptide according to claim 21 comprising the steps of:
(a) providing a peptide sequence consisting of a T-cell epitope of said antigenic protein, and
(b) linking to said peptide sequence said oxidoreductase motif, such that said motif and said epitope are either adjacent to each other or separated by a linker of between 0 and 7 amino acids.

32. An in vitro method for obtaining a population of antigen-specific cytolytic CD4+ T cells, against antigen presenting cells (APC) presenting said antigen, the method comprising the steps of:
providing peripheral blood cells,
contacting said peripheral blood cells with an immunogenic peptide according to claim 21; and
expanding said peripheral blood cells in the presence of IL-2.

33. An in vitro method for obtaining a population of antigen-specific NKT cells, the method comprising the steps of:
providing peripheral blood cells,
contacting said peripheral blood cells with an immunogenic peptide according to claim 21; and
expanding said peripheral blood cells in the presence of IL-2.

34. A population of antigen-specific cytolytic CD4+ T cells or antigen-specific NKT cells, wherein the antigen-specific cytolytic CD4+ T cells or antigen-specific NKT cells are produced by a method comprising the steps of:
providing peripheral blood cells,
contacting said peripheral blood cells with an immunogenic peptide according to claim 21; and
expanding said peripheral blood cells in the presence of IL-2.

35. A method of treating an autoimmune disease, an infection with an intracellular pathogen, a tumor, an allograft rejection, or suppressing an immune response to a soluble allofactor, allergen exposure or viral vector used for gene therapy or gene vaccination in a subject in need thereof comprising administering to the subject a therapeutically effective amount of the population of antigen-specific cytolytic CD4+ T cells or antigen-specific NKT cells according to claim 34.

36. A method for obtaining a population of antigen-specific cytolytic CD4+ T cells, against antigen presenting cells (APC) presenting said antigen, the method comprising the steps of:
providing an immunogenic peptide according to claim 21;
administering said peptide to a subject; and
obtaining said population of antigen-specific cytolytic CD4+ T cells from said subject.

37. A method for obtaining a population of antigen-specific NKT cells, the method comprising the steps of:
providing an immunogenic peptide according to claim 21;
administering said peptide to a subject; and
obtaining said population of antigen-specific NKT cells from said subject.

38. A population of antigen-specific cytolytic CD4+ T cells or antigen-specific NKT cells, wherein
a) the antigen-specific cytolytic CD4+ T cells are produced by a method comprising the steps of:
providing an immunogenic peptide according to claim 21;
administering said peptide to a subject; and
obtaining said population of antigen-specific cytolytic CD4+ T cells from said subject; and
b) the antigen-specific NKT cells are produced by a method comprising the steps of:
providing an immunogenic peptide according to claim 21;
administering said peptide to a subject; and
obtaining said population of antigen-specific NKT cells from said subject.

39. A method of treating an autoimmune disease, an infection with an intracellular pathogen, a tumor, an allograft rejection, or suppressing an immune response to a soluble allofactor, allergen exposure or viral vector used for gene therapy or gene vaccination in a subject in need thereof comprising administering to the subject a therapeutically effective amount of the population of antigen-specific cytolytic CD4+ T cells or antigen-specific NKT cells according to claim 38.

40. A polynucleotide encoding the immunogenic peptide according to claim 21.
